(19) United States
(12) Patent Application Publication
(10) Pub. No.: US 2022/0330599 A1
(43) Pub. Date: Oct. 20, 2022

(54) Microbial Protein Hydrolysate Compositions and Methods of Making Same

(71) Applicant: Kiverdi, Inc., Pleasanton (US)

(72) Inventors: Kenneth C. Zahn, Pleasanton, CA (US); John S. Reed, Pleasanton, CA (US); Sonali P. Hande, Pleasanton, CA (US); Chloe Bantock, Pleasanton, CA (US); Kripa K. Rao, Pleasanton, CA (US)

(21) Appl. No.: 17/760,934

(22) PCT Filed: Sep. 15, 2020
(86) PCT No.: PCT/US20/50902

§ 371 Date: Mar. 16, 2022

Related U.S. Application Data
(60) Provisional application No. 62/901,169, filed on Sep. 16, 2019.
(60) Provisional application No. 62/943,754, filed on Dec. 4, 2019.

(51) Int. Cl. A23L 33/18; A23L 33/135; A01N 63/50

(52) U.S. Cl. CPC A23L 33/18; A23L 33/135; A01N 63/50

(57) ABSTRACT

Protein hydrolysate compositions and methods of making the same are disclosed. The protein hydrolysate composition has a protein-rich organic content. The protein hydrolysate composition may be substantially free of exogenous chelating agents, chaotropic agents and surfactants. The protein hydrolysate composition may be low in ash content. The protein hydrolysate composition is produced by processing a biomass, e.g., a microbial biomass, through a combination of physical, chemical and/or enzymatic treatments. The protein hydrolysate may be sourced via microbial biomass from CCk as a carbon source. Also disclosed are methods of using the protein hydrolysate compositions, e.g., as a biostimulant.

### CROSS-REFERENCE TO RELATED APPLICATIONS

This application claims the benefit of U.S. Provisional Application No. 62/901,169, filed Sep. 16, 2019, and U.S. Provisional Application No. 62/943,754, filed Dec. 4, 2019, both of which are incorporated herein by reference in their entireties.

### FIELD OF INVENTION

The present disclosure relates to the fields of protein hydrolysates produced from biological sources, and methods of making the same and formulating the same into various end products. In particular, the present disclosure relates to a novel process for converting protein containing biomass produced from renewable sources, such as biological processes designed to capture carbon dioxide emissions and other waste carbon conversion or diversion processes, into protein hydrolysates.

### BACKGROUND

Protein is a nutrient that humans and animals need to grow and to support and maintain life. As a result, protein is an important component of many foods and animal feed. In addition, proteins and protein hydrolysates can serve as a biostimulant or plant nutrient to promote the growth of plants, e.g., in soil. Furthermore, proteins and protein hydrolysates can be used as a nutrient source for the growth of fungi or microorganisms. Protein hydrolysates have also been found to stimulate the growth and activity of beneficial microorganisms in the plant microbiome and in the soil. Protein that has been hydrolyzed (i.e., protein hydrolysates) have been found to be particularly useful as a source of nutrition. This process breaks the protein molecule down into smaller peptides and amino acid molecules, which are more easily digested and absorbed.

Microbially produced proteins or protein containing biomass may be particularly useful for producing protein hydrolysates for consumption and/or use as biostimulants. Microorganisms may be used to convert a carbon and nitrogen containing feedstock into proteins or protein containing biomass. Chemoautotrophic microorganisms may be used beneficially to capture carbon dioxide from the atmosphere or from a point source of carbon dioxide emissions or from a conversion process producing carbon dioxide. More value may be gained from carbon fixing processes by producing valuable products from these microorganism cultures.

Various prior art methods exist for hydrolyzing proteins, including various chemical and enzymatic methods. Such prior art methods are not fully effective for a variety of reasons, including the inability to achieve the preferred level of hydrolysis for a consumable or biostimulant product, the presence of detrimental residual chemicals or side products for such products, inability or impracticality of scaling the process up to a larger-scale commercial process and the inability to use highly scalable feedstocks such as CO₂.

### SUMMARY OF THE INVENTION

In one aspect the disclosure provides for a process for producing a biologically derived protein hydrolysate. In accordance with certain embodiments, the process comprises the steps of: (1) culturing a microorganism in the presence of a carbon source in an aerobic or microaerobic bioprocess to grow biomass containing protein, wherein the microorganism comprises Cupriavidus necator and the carbon source comprises carbon dioxide; (2) harvesting the biomass, which contains protein, into a suspension composition; (3) if the pH of the suspension composition is not within a first target pH range, adjusting the pH of the suspension composition to a pH within the first target pH range, and wherein the first target pH range is at least about 10, thereby forming an alkaline suspension composition; (4) heating the alkaline suspension composition to a first temperature of at least about 40° C. for a first time period of at least about 5 minutes; (5) forming a neutralized suspension composition by adding a neutralizing agent to the alkaline suspension composition, wherein the pH of the neutralized suspension composition is within a second target pH range, and wherein the second target pH range is from about 6.5 to about 9.5; (6) optionally further hydrolyzing the proteins by adding a protease to the neutralized suspension composition and incubating the neutralized suspension composition at a second temperature range for a second time period, wherein the second temperature range is at least about 40° C. and the second time period is at least about 1 hour, thereby forming a hydrolyzed protein suspension; and (7) capturing the supernatant containing the hydrolyzed protein from the hydrolyzed protein suspension.

In some embodiments, the process includes: (a) adjusting the pH of a biomass suspension composition to a pH within a first target pH range of at least about 10, if needed, wherein the biomass suspension or the pH adjusted suspension composition produced in step (a) is an alkaline suspension composition; and (b) heating the alkaline suspension composition to a first temperature of at least about 40° C. for a first time period of at least about 5 minutes, thereby producing an alkaline hydrolysate suspension that comprises hydrolyzed microbial protein. In some embodiments, the process further includes, after step (b): (i) neutralizing the alkaline suspension composition by adding a neutralizing agent to the alkaline suspension composition, thereby forming a neutralized suspension composition, wherein the pH of the neutralized suspension composition is within a second target pH range of about 6.5 to about 9.5; and (ii) adding a protease to the neutralized suspension composition at a second temperature of at least about 40° C. and a second time period of at least about 1 hour, thereby further hydrolyzing the microbial protein and forming a protease hydrolysate suspension that comprises hydrolyzed microbial protein. In certain embodiments, the process further includes: (c) separating a liquid supernatant from solid material in the alkaline hydrolysate suspension or he protease hydrolysate suspension, wherein the supernatant comprises soluble hydrolyzed microbial protein. In certain embodiments, step (b) includes application of pressure of at least about 15 psi to the alkaline suspension for at least a portion of the first time period. For example, adjusting the pH in step (b) may include adding one or more base selected from potassium hydroxide, ammonium hydroxide, ammonia, calcium hydroxide, and sodium hydroxide.

In accordance with certain embodiments, the process comprises the steps of: (1) culturing a microorganism in the presence of a carbon source in an aerobic or microaerobic bioprocess to grow biomass containing protein, wherein the microorganism comprises Cupriavidus necator and the carbon source comprises carbon dioxide; (2) harvesting the biomass, which contains protein, into a suspension composition; (3) if the pH of the suspension composition is not within a first target pH range, adjusting the pH of the suspension composition to a pH within the first target pH range, and wherein the first target pH range is no more than about 0.5 to about 3, thereby forming an acidic suspension composition; (4) heating the acidic suspension composition to a first temperature of at least about 40° C. for a first time period of at least about 5 minutes; (5) forming a neutralized suspension composition by adding a neutralizing agent to the acidic suspension composition, wherein the pH of the neutralized suspension composition is within a second target pH range, and wherein the second target pH range is from about 5 to about 6.5 or about 8.0; (6) optionally further hydrolyzing the proteins by adding a protease to the neutralized suspension composition and incubating the neutralized suspension composition at a second temperature range for a second time period, wherein the second temperature range is at least about 40° C. and the second time period is at least about 1 hour, thereby forming a hydrolyzed protein suspension; and (7) capturing the supernatant containing the hydrolyzed protein from the hydrolyzed protein suspension.

In some embodiments, the process includes: (a) adjusting the pH of a biomass suspension composition to a pH within a first target pH range of about 0.5 to about 3, if needed, wherein the biomass suspension or the pH adjusted suspension composition produced in step (a) is an acidic suspension composition; and (b) heating the acidic suspension composition to a first temperature of at least about 40° C. for a first time period of at least about 5 minutes, thereby producing an acidic hydrolysate suspension that comprises hydrolyzed microbial protein. In some embodiments, the process includes, after step (b): (i) neutralizing the acidic suspension by adding a neutralizing agent to the acidic suspension composition, thereby forming a neutralized suspension composition, wherein the pH of the neutralized suspension composition is within a second target pH range of about 5 to about 8; and (ii) adding a protease to the neutralized suspension composition at a second temperature of at least about 40° C. and a second time period of at least about 1 hour, thereby further hydrolyzing the microbial protein and forming a protease hydrolysate suspension that comprises hydrolyzed microbial protein. In certain embodiments, the process further includes: (c) separating a liquid supernatant from solid material in the acidic hydrolysate suspension or the protease hydrolysate suspension, wherein the supernatant comprises soluble hydrolyzed microbial protein. In certain embodiments, step (b) includes application of pressure of at least about 15 psi to the acidic suspension for at least a portion of the first time period. In certain embodiments, adjusting the pH in step (b) may include adding one or more acid selected from phosphoric acid, sulfuric acid, nitric acid, formic acid, acetic acid, carbonic acid, and hydrochloric acid.

In accordance with certain embodiments, the process comprises the steps of: (1) culturing a microorganism to grow biomass; (2) harvesting the biomass, which contains protein, in a suspension composition; (3) adjusting the pH of the suspension composition, if necessary, to a pH of at least about 10, and preferably to a pH of about 10 to about 12; (4) optionally, adding a chelating agent and/or a surfactant to the composition; (5) heating the composition to at least about 40° C. for at least 10 minutes, and preferably between about 40° C. to about 130° C. for about 10 minutes to about 8 hours, and optionally pressurizing the composition to a pressure of greater than about 15 psi during at least a portion of the heating, and preferably to a pressure of about 20 psi to about 50 psi; (6) applying a neutralizing agent to the composition to adjust the pH to within a range from about 7.5 to about 9.5, and preferably from about 8.5 to about 9; (7) optionally adding a protease to the composition which further hydrolyzes the protein; and (8) capturing the supernatant containing the hydrolyzed protein from the suspension; and (9) optionally, drying the supernatant and lyophilizing the hydrolyzed protein.

In accordance with certain embodiments, the process comprises the steps of: (1) culturing a microorganism to grow biomass; (2) harvesting the biomass, which contains protein, in a suspension composition; (3) adjusting the pH of the suspension composition, if necessary, to a pH of no more than about 3, and preferably to a pH of about 1 to about 1.5; (4) optionally, adding a chelating agent and/or a surfactant to the composition; (5) heating the composition to at least about 40° C. for at least 10 minutes, and preferably between about 40° C. to about 130° C. for about 10 minutes to about 8 hours, and optionally pressurizing the composition to a pressure of greater than about 15 psi during at least a portion of the heating, and preferably to a pressure of about 20 psi to about 50 psi; (6) applying a neutralizing agent to the composition to adjust the pH to within a range from about 5 to about 7, and preferably from about 6 to about 6.5; (7) optionally adding a protease to the composition which further hydrolyzes the protein; and (8) capturing the supernatant containing the hydrolyzed protein from the suspension; and (9) optionally, drying the supernatant and lyophilizing the hydrolyzed protein.

In certain embodiments, any of the processes described herein may further include formulation of the hydrolyzed microbial protein for use as a biostimulant. In certain embodiments, any of the processes described herein may further include application of the hydrolyzed microbial protein, or a composition or formulation thereof, to seeds, plants, or soil, wherein a plant grown in contact with the composition exhibits greater growth in the presence of the composition than in the absence of the composition.

Also disclosed herein are protein hydrolysate compositions derived from proteins or a protein containing source or proteinaceous materials, and methods of making the same. The present disclosure includes protein hydrolysate compositions derived from a microbial source and methods of making the same. The protein hydrolysate composition has a protein-rich organic content. In some embodiments, the protein hydrolysate composition is substantially free of exogenous chelating agents, chaotropic agents and/or surfactants. The protein hydrolysate compositions of the present disclosure may have various agricultural or horticultural uses, for example, as a biostimulant. The protein hydrolysate compositions of the present disclosure may find use in nutritional or medicinal applications for animals and humans. The protein hydrolysate compositions of the present disclosure may find use as nutritional source for cells, including prokaryotic and eukaryotic cells. A source of the protein hydrolysate of the present disclosure includes microbial sources, including photoautotrophic, chemoautotrophic or oxyhydrogen microbe cultures grown in, e.g., a bioreactor. The bioreactor may be configured to use waste or low value sources of carbon, such as CO₂, to culture the oxyhydrogen microbe. Thus, the protein hydrolysate compositions of the present disclosure may be sustainably produced from waste or low value sources of carbon, such as CO₂. The protein hydrolysate composition may be sustainably produced from CO₂, CH₄, CO, and/or other carbon containing gases that are greenhouse gases (GHGs) or sources of pollution, e.g., air pollution.

Methods of producing the protein hydrolysate composition are also disclosed. The method includes processing a proteinaceous material with a combination of physical, chemical and/or enzymatic treatments. The proteinaceous material may include a cellular biomass, such as a microbial biomass. The method may include subjecting a suspension of a proteinaceous material, e.g., a cellular biomass, having a pH of about 11.0 or higher or having a pH of about 3 or lower to a temperature of at least about 40° C. for a suitable amount of time. In some embodiments, the method includes subjecting the alkaline or acidic biomass suspension to a super-atmospheric pressure. In certain embodiments, the heat treatment generates an extracted suspension, and the method includes contacting the extracted suspension with a neutralizing buffer to reduce the pH to 9.5 or lower (for alkaline hydrolysis conditions) or to increase the pH to about 6 or higher (for acidic hydrolysis conditions), to generate a neutralized suspension; and optionally, contacting the neutralized suspension with a protease, to produce a protein hydrolysate composition in a soluble fraction. In some embodiments, the protease is an alkaline protease. In some embodiments, the protease is an acidic protease. In some embodiments, the protease is a metalloprotease.

In some embodiments, a protein hydrolysate composition of the present disclosure has a protein-rich organic content, wherein the composition is substantially free of exogenous chelating agents, chaotropic agents and surfactants. In certain embodiments, the protein hydrolysate is of microbial origin. In certain embodiments, the composition is substantially free of sodium and/or chloride. In certain embodiments, the protein hydrolysate composition has a size distribution of polypeptides of 25 kD or smaller. In some embodiments, the total nitrogen content of the composition is about 5% (w/w) or more. In some embodiments, the phosphate content of the composition is about 5% (w/w) or more. In some embodiments, the potassium content of the composition is about 5% (w/w) or more. In some embodiments, the sodium content of the composition is about 1% (w/w) or less. In some embodiments, the chloride content of the composition is about 1% (w/w) or less.

In some embodiments, the protein hydrolysate composition is lyophilized.

A method of producing a protein hydrolysate composition of the present disclosure includes adjusting the pH of a biomass suspension to 11.0 or higher to generate an alkaline suspension, and subjecting the alkaline suspension to a temperature of at least about 40° C., under conditions sufficient to generate a protein hydrolysate composition. In some embodiments, the method includes subjecting the alkaline suspension to a super-atmospheric pressure.

In certain embodiments, the method includes neutralizing the suspension to pH 9.5 or lower after subjecting the alkaline suspension to the temperature of at least about 40° C., to generate a neutralized suspension, and contacting the neutralized suspension with a protease, to produce a protein hydrolysate composition in a soluble fraction of the suspension. In some embodiments, the protease is an alkaline protease. In some embodiments, the method includes clarifying the suspension; and lyophilizing the soluble fraction.

In some embodiments, the method includes contacting a suspension comprising a biomass with a base to adjust the pH, where the base includes one or more of potassium hydroxide, calcium hydroxide, calcium oxide, ammonium hydroxide or ammonia.

A method of producing a protein hydrolysate composition of the present disclosure includes adjusting the pH of a biomass suspension to 3 or lower to generate an acidic suspension, and subjecting the acidic suspension to a temperature of at least about 40° C., under conditions sufficient to generate a protein hydrolysate composition. In some embodiments, the method includes subjecting the acidic suspension to a super-atmospheric pressure.

In certain embodiments, the method includes neutralizing the suspension to about pH 5 or higher after subjecting the acidic suspension to the temperature of at least about 40° C., to generate a neutralized suspension, and contacting the neutralized suspension with a protease, to produce a protein hydrolysate composition in a soluble fraction of the suspension. In some embodiments, the protease is an acidic protease. In some embodiments, the method includes clarifying the suspension; and lyophilizing the soluble fraction.

In some embodiments, the method includes contacting a suspension comprising a biomass with an acid to adjust the pH, where the acid includes phosphoric acid or sulfuric acid.

In some embodiments, the biomass is a microbial biomass.

In some embodiments, the microbial biomass suspension is subjected to the temperature and/or temperature plus super-atmospheric pressure for a time period from about 5 to about 90 minutes.

In some embodiments, the protein hydrolysate composition is substantially free of exogenous chelating agents, chaotropic agents and/or surfactants. In some embodiments, the protein hydrolysate composition includes a protein-rich organic content. In some embodiments, the protein hydrolysate composition includes a nitrogen, phosphorus, and potassium (NPK) content of at least 5% (w/w) of each element. In some embodiments, the protein hydrolysate composition is substantially free of sodium and/or chloride.

In some embodiments, the method includes separating an insoluble fraction of the suspension from the soluble fraction; and extracting a polymeric composition from the insoluble fraction. In some embodiments, the polymeric composition is a polyhydroxyalkanoate (PHA), for example, a polyhydroxybutyrate (PHB) composition.

Also provided is a protein hydrolysate composition made using a method of the present disclosure. A plant supplement that includes a protein hydrolysate composition of the present disclosure is also provided. The plant supplement may be applied to a plant to, e.g., provide nutrients and/or promote growth. The protein hydrolysate composition of the present disclosure also finds use as a nutritional supplement, e.g., for animals or cells.

In one aspect, a method is provided for producing a protein hydrolysate comprising the steps of: culturing a microorganism in the presence of a carbon source in an aerobic or microaerobic bioprocess to grow biomass containing protein, wherein the microorganism comprises Cupriavidus necator and the carbon source comprises carbon dioxide; harvesting the biomass, which includes protein, into a suspension composition; if the pH of the suspension composition is not within a first target pH range, adjusting the pH of the suspension composition to a pH within the first target pH range, and wherein the first target pH range is at least about 10, thereby forming an alkaline suspension composition; heating the alkaline suspension composition to a first temperature of at least about 40° C. for a first time period of at least about 5 minutes; forming a neutralized suspension composition by adding a neutralizing agent to the alkaline suspension composition, wherein the pH of the neutralized suspension composition is within a second target pH range, and wherein the second target pH range is from about 6.5 to about 9.5; forming a hydrolyzed protein suspension by adding a protease to the neutralized suspension composition and incubating the neutralized suspension composition at a second temperature range for a second time period, wherein the second temperature range is at least about 40° C. and the second time period is at least about 1 hour; and capturing the supernatant containing the hydrolyzed protein from the hydrolyzed protein suspension.

In one embodiment, the step of harvesting protein from the biomass in a suspension composition comprises suspending the biomass in a biocompatible liquid medium. For example, the biocompatible liquid medium may comprise water or a buffer. In some embodiments, the biomass is suspended in the liquid medium by vortexing, homogenizing, stirring, or sonicating.

In some embodiments, the first target pH range is at least about 11, or about 10 to about 13, or about 10.5 to about 13, or about 10.5 to about 12.5, or about 10.5 to about 11.5.

In some embodiments, the step of adjusting the pH of the suspension composition to the first target pH range comprises adding a base to the suspension composition, wherein the base comprises one or more of potassium hydroxide (KOH), ammonium hydroxide (NH₄OH), ammonia (NH₃), calcium hydroxide (Ca(OH)₂), and sodium hydroxide (NaOH), e.g., comprises potassium hydroxide (KOH), ammonium hydroxide (NH₄OH), ammonia (NH₃), calcium hydroxide (Ca(OH)₂), and/or sodium hydroxide (NaOH), e.g., selected from the group consisting of potassium hydroxide (KOH), ammonium hydroxide (NH₄OH), ammonia (NH₃), calcium hydroxide (Ca(OH)₂), and sodium hydroxide (NaOH).

In some embodiments, the second target pH range is from about 7 to about 9.5, from about 8 to about 9.5, from about 8.5 to about 9.5, or from about 9 to about 9.5.

In some embodiments, the neutralizing agent comprises one or more of potassium phosphate, ammonium phosphate, sodium citrate, citric acid, sodium phosphate, phosphoric acid, phosphate buffer, Tris, HEPES, glycine, carbon dioxide, bicarbonate, and carbonic acid, e.g., comprises potassium phosphate, ammonium phosphate, sodium citrate, citric acid sodium phosphate, phosphoric acid, phosphate buffer, Tris, HEPES, glycine, carbon dioxide, bicarbonate, and/or carbonic acid, e.g., selected from the group consisting of potassium phosphate, ammonium phosphate, phosphoric acid, phosphate buffer, sodium citrate, citric acid, sodium phosphate, Tris, HEPES, glycine, carbon dioxide, bicarbonate, and carbonic acid.

In one aspect, a method is provided for producing a protein hydrolysate comprising the steps of: culturing a microorganism in the presence of a carbon source in an aerobic or microaerobic bioprocess to grow biomass containing protein, wherein the microorganism comprises Cupriavidus necator and the carbon source comprises carbon dioxide; harvesting the biomass, which contains protein, into a suspension composition; if the pH of the suspension composition is not within a first target pH range, adjusting the pH of the suspension composition to a pH within the first target pH range, and wherein the first target pH range is no more than about 0.5 to about 3, thereby forming an acidic suspension composition; heating the acidic suspension composition to a first temperature of at least about 40° C. for a first time period of at least about 5 minutes; forming a neutralized suspension composition by adding a neutralizing agent to the acidic suspension composition, wherein the pH of the neutralized suspension composition is within a second target pH range, and wherein the second target pH range is from about 5 to about 6.5, or about 5 to about 8; optionally further hydrolyzing the protein suspension by adding a protease to the neutralized suspension composition and incubating the neutralized suspension composition at a second temperature range for a second time period, wherein the second temperature range is at least about 40° C. and the second time period is at least about 1 hour; and capturing the supernatant containing the hydrolyzed protein from the hydrolyzed protein suspension.

In one embodiment, the step of harvesting protein from the biomass in a suspension composition comprises suspending the biomass in a biocompatible liquid medium. For example, the biocompatible liquid medium may comprise water or a buffer. In some embodiments, the biomass is suspended in the liquid medium by vortexing, homogenizing, stirring, or sonicating.

In some embodiments, the first target pH range is at no more than about 3, or about 0.5 to about 3.

In some embodiments, the step of adjusting the pH of the suspension composition to the first target pH range comprises adding an acid to the suspension composition, wherein the acid comprises one or more of phosphoric acid (H₃PO₄), sulfuric acid (H₂SO₄), nitric acid (HNO₃), formic acid (HCOOH), acetic acid (CH₃COOH), hydrochloric acid (HCl), and carbonic acid, i.e., carbon dioxide (CO₂).

In some embodiments, the second target pH range is from about 3 to about 6, or about 3 to about 9.

In some embodiments, the neutralizing agent comprises one or more of calcium hydroxide (Ca(OH)₂), calcium oxide, calcium carbonate (CaCO₃), potassium hydroxide (KOH), phosphate buffer, ammonium bicarbonate, ammonium carbonate, ammonium hydroxide (NH₄OH), and ammonia.

In some embodiments, the protein hydrolysate is used as a biostimulant and/or plant nutrient (e.g., nutrient that is essential or beneficial for plant growth and/or metabolism), or precursor thereof, and the step of adjusting the pH of the suspension composition to the first target pH range comprises adding a base (for alkaline hydrolysis condition) or an acid (for acid hydrolysis conditions) to the suspension composition, wherein the base or acid comprises plant nutrients comprising one or more of from nitrogen (N), phosphorus (P), and potassium (K), e.g., comprises from nitrogen (N), phosphorus (P), and/or potassium (K), e.g., selected from the group consisting of nitrogen (N), phosphorus (P), and potassium (K). In some embodiments, the protein hydrolysate is used as a biostimulant and/or plant nutrient, or precursor thereof, and the step of adjusting the pH of the suspension composition to the first target pH range comprises adding a base or acid to the suspension composition, wherein the base or acid does not contain elements that are harmful to plant growth. In some embodiments, the protein hydrolysate is used as a biostimulant and/or plant nutrient, or precursor thereof, and the step of adjusting the pH of the suspension composition to the first target pH range comprises adding a base or acid to the suspension composition, wherein the base does not contain sodium or chloride at levels which inhibit plant growth.

In some embodiments, the method further comprises the step of adding a chelating agent and/or surfactant to the suspension composition after the step of adjusting the pH of the suspension composition to the first target pH range. For example, a chelating agent may be added that comprises one or more of ethylenediaminetetraacetic acid (EDTA) and ethylene glycol-bis(β-aminoethyl ether)-N,N,N′,N′-tetraacetic acid (EGTA), e.g., comprises ethylenediaminetetraacetic acid (EDTA) and/or ethylene glycol-bis(β-aminoethyl ether)-N,N,N′,N′-tetraacetic acid (EGTA), e.g., selected from the group consisting of ethylenediaminetetraacetic acid (EDTA) and ethylene glycol-bis(β-aminoethyl ether)-N,N,N′,N′-tetraacetic acid (EGTA). In some embodiments, the amount of the chelating agent added to the suspension composition is in the range of about 0.1 mM to about 10 mM, or about 0.5 mM to about 8 mM, or about 1 mM to about 7 mM, or about 3 mM to about 5 mM. In some embodiments, the protein hydrolysate is used as a biostimulant and/or plant nutrient, or precursor thereof, and the chelating agent and/or surfactant comprises plant nutrients that comprise one or more of nitrogen (N), phosphorus (P), and potassium (K), e.g., comprises nitrogen (N), phosphorus (P), and/or potassium (K), e.g., selected from the group consisting of nitrogen (N), phosphorus (P), and potassium (K). In some embodiments, the chelating agent and/or surfactant does not contain elements that are harmful to plant growth. For example, a surfactant may be added that comprises one or more of sodium dodecyl sulfate (SDS), ammonium lauryl sulfate, Triton X-100, Tween 80, Tween 20, and Pluronic PF-68, e.g., comprises sodium dodecyl sulfate (SDS), ammonium lauryl sulfate, Triton X-100, Tween 80, Tween 20, and/or Pluronic PF-68, e.g., selected from the group consisting of sodium dodecyl sulfate (SDS), ammonium lauryl sulfate, Triton X-100, Tween 80, Tween 20, and Pluronic PF-68. In some embodiments, the amount of the surfactant added to the suspension composition relative to the dry weight of biomass in the suspension is in the range of about 1% to about 25%, or about 2% to about 20%, or about 4% to about 15%, or about 5% to about 12%, or about 8% to about 12%. In some embodiments, the surfactant is non-toxic to plants or animals, and/or is biodegradable.

In some embodiments, the alkaline or acidic suspension composition is heated to a first temperature of from about 40° C. to about 150° C. for a first time period of from about 5 minutes to about 24 hours. For example, the first temperature may be at least about 50° C., at least about 60° C., at least about 70° C., at least about 80° C., at least about 90° C., at least about 100° C., at least about 105° C., at least about 110° C., from about 40° C. to about 150° C., from about 60° C. to about 150° C., from about 70° C. to about 140° C., from about 80° C. to about 140° C., from about 90° C. to about 135° C., from about 100° C. to about 130° C., from about 100° C. to about 125° C., from about 105° C. to about 125° C., or from about 110° C. to about 125° C. For example, the first time period may be at least about 10 minutes, at least about 15 minutes, at least about 20 minutes, at least about 25 minutes, at least about 30 minutes, at least about 45 minutes, at least about 60 minutes, at least about 75 minutes, at least about 90 minutes, at least about 3 hours, at least about 5 hours, at least about 8 hours, at least about 12 hours, at least about 16 hours, at least about 20 hours, at least about 24 hours, from about 5 minutes to about 90 minutes, from about 5 minutes to about 80 minutes, from about 5 minutes to 70 minutes, from about 10 minutes to about 60 minutes, from about 10 minutes to about 50 minutes, from about 10 to about 40 minutes, from about 10 to about 30 minutes, from about 20 to about 60 minutes, from about 30 to about 60 minutes, from about 1 hour to about 24 hours, from about 1 hour to about 18 hours, from about 1 hour to about 12 hours, or from about 1 hour to about 8 hours. In some embodiments, the step of heating the alkaline or acidic suspension composition to a first temperature is done with the alkaline or acidic suspension under pressure. For example, the pressure of the alkaline or acidic suspension during at least a portion of the first time period may be at least about 15 psi, at least about 20 psi, at least about 25 psi, at least about 30 psi, at least about 35 psi, at least about 40 psi, at least about 45 psi, from about 15 psi to about 50 psi, from about 15 psi to about 40 psi, from about 15 psi to about 35 psi, from about 15 psi to about 30 psi, from about 15 psi to about 25 psi, or from about 15 psi to about 20 psi. In some embodiments, the pressure of the alkaline or acidic suspension during at least a portion of the first time period is at least about 15 psi, at least about 20 psi, at least about 25 psi, at least about 30 psi, at least about 35 psi, at least about 40 psi, at least about 45 psi, from about 15 psi to about 50 psi, from about 15 psi to about 40 psi, from about 15 psi to about 35 psi, from about 15 psi to about 30 psi, from about 15 psi to about 25 psi, or from about 15 psi to about 20 psi, and wherein said portion of the first time period comprises at least about 50%, at least about 55%, at least about 60%, at least about 65%, at least about 70%, at least about 75%, at least about 80%, at least about 85%, at least about 90%, or at least about 95% of said first time period.

In some embodiments, the protein hydrolysate is used as a biostimulant and/or plant nutrient, or precursor thereof, and the neutralizing agent comprises plant nutrients that comprise one or more of nitrogen (N), phosphorus (P), and potassium (K), e.g., comprise nitrogen (N), phosphorus (P), and potassium (K), e.g., selected from the group consisting of nitrogen (N), phosphorus (P), and potassium (K). In some embodiments, the protein hydrolysate is used as a biostimulant and/or plant nutrient, or precursor thereof, and the neutralizing agent does not contain elements that are harmful to plant growth.

In some embodiments, the method further comprises the additional step of adding a chaotropic agent to the neutralized suspension composition. In some embodiments, the chaotropic agent is added before or with a protease such that the protease hydrolysis is carried out in the presence of the chaotropic agent. For example, the chaotropic agent may comprise one or more of urea, thiourea and guanidium chloride, e.g., comprises urea, thiourea and/or guanidium chloride, e.g., selected from the group consisting of urea, thiourea and guanidium chloride. In some embodiments, the chaotropic agent is added in an amount in the range of about 0.1 M to about 2 M, about 0.5 M to about 1.5 M, or about 0.8 M to about 1.2 M. In some embodiments, the protein hydrolysate is used as a biostimulant and/or plant nutrient, or precursor thereof, and the chaotropic agent comprises plant nutrients that comprise one or more of nitrogen (N), phosphorus (P), and potassium (K), e.g., comprises nitrogen (N), phosphorus (P), and/or potassium (K), e.g., selected from the group consisting of nitrogen (N), phosphorus (P), and potassium (K). In some embodiments, the protein hydrolysate is used as a biostimulant and/or plant nutrient, or precursor thereof, and the chaotropic agent does not contain elements that are harmful to plant growth.

In some embodiments, the protease comprises one or more of an endoproteinase, an exoproteinase, an alkaline protease, a serine alkaline protease, a bacterial alkaline protease, and subtilisin A, e.g., comprises an endoproteinase, an exoproteinase, an alkaline protease, a serine alkaline protease, a bacterial alkaline protease, subtilisin A, e.g., selected from the group consisting of an endoproteinase, an exoproteinase, an alkaline protease, a serine alkaline protease, a bacterial alkaline protease, and subtilisin A. In other embodiments, the protease comprises an acidic protease. In further embodiments, the protease comprises a metalloprotease.

In some embodiments, the second temperature range is from about 40° C. to about 70° C., from about 45° C. to about 65° C. or from about 50° C. to about 60° C.

In some embodiments, the second time period is for a minimum of about 1 hour, about 3 hours, about 6 hours, about 12 hours, about 18 hours, or about 24 hours, and a maximum of about 4 hours, about 8 hours, about 12 hours, about 24 hours, about 36 hours, or about 48 hours.

In some embodiments, at least 75% or at least 80% or at least 85% or at least 90% or at least 95% of the polypeptides in the captured supernatant have an atomic mass of less than about 25 kD, less than about 20 kD, less than about 15 kD, less than about 10 kD, less than about 5 kD, less than about 3 kD, less than about 2 kD, from about 0.1 kD to about 30 kD, from about 1 kD to about 30 kD, from about 1 kD to about 25 kD, from about 5 kD to about 25 kD, from about 0.1 kD to about 2 kD, from about 0.1 kD to about 5 kD, from about 0.1 kD to about 10 kD, or from about 5 kD to about 20 kD.

In some embodiments, the method further comprises the additional step of clarifying the suspension through centrifugation or filtration to remove undissolved material in the hydrolyzed protein suspension.

In some embodiments, the method further comprises the additional step of clarifying the suspension or solution, for example, via filtration (e.g., ultrafiltration), to remove molecules above a certain molecular weight (MW) cut-off. In some such embodiments, the MW cut-off may be greater than about 20 kD, greater than about 15 kD, greater than about 10 kD, greater than about 5 kD, or greater than about 2 kD.

In some embodiments, the method further comprises the additional step of drying the captured supernatant and lyophilizing the hydrolyzed protein. For example, the lyophilized protein hydrolysate composition may have a water content from about 1% to about 10%, about 1% to about 8%, about 1% to about 6%, or about 2% to about 5%.

In some embodiments, the method further comprises the steps of: (1) adding a chelating agent and/or surfactant and/or chaotropic agent to the suspension composition after the step of adjusting the pH of the suspension composition to the first target pH range; and/or (2) adding a chaotropic agent to the neutralized suspension composition; and (3) removing substantially all of the surfactant, chelating agent and/or chaotropic agent after formation of the hydrolyzed protein suspension. In some embodiments, the surfactant, chelating agent and/or chaotropic agent are removed through gel filtration chromatography, membrane filtration, and/or dialysis.

In some embodiments, at least a portion of the protein is hydrolyzed by the step of heating the alkaline or acidic suspension composition to a first temperature for a first time period.

In some embodiments, the suspension composition comprises a lysate.

In some embodiments, the step of harvesting the protein from the biomass into a suspension composition comprises subjecting the biomass to lysis.

### BRIEF DESCRIPTION OF THE DRAWINGS

FIG. 1A is a schematic diagram of a method for producing a protein hydrolysate, according to embodiments of the present disclosure.

FIG. 1B is a schematic diagram of a method for producing a protein hydrolysate, according to embodiments of the present disclosure.

FIG. 2 is a schematic diagram of a method for producing a protein hydrolysate, according to embodiments of the present disclosure.

FIG. 3A is a schematic diagram of a method for producing a protein hydrolysate, according to embodiments of the present disclosure.

FIG. 3B is a schematic diagram of a method for producing a protein hydrolysate, according to embodiments of the present disclosure.

FIG. 4 is a schematic diagram of a method for producing a protein hydrolysate, according to embodiments of the present disclosure.

FIG. 5 is a schematic diagram of an integrated process for producing a protein hydrolysate from a microbial culture system.

FIG. 6 shows a protein gel analysis of protein hydrolysis products prepared according to embodiments of the present disclosure.

FIG. 7 shows a protein gel analysis of the fractionation of protein hydrolysis products which have not been subjected to an enzymatic hydrolysis step.

FIG. 8A shows a protein gel analysis of protein hydrolysis products prepared according to embodiments of the present disclosure.

FIG. 8B shows a protein gel analysis of protein hydrolysis products prepared according to embodiments of the present disclosure.

FIG. 9 shows a protein gel analysis of protein hydrolysis products prepared according to embodiments of the present disclosure.

FIG. 10 shows from left to right: Turnips treated with: (a) water; (b) NPK fertilizer; (c) protein hydrolysate.

FIG. 11 shows from left to right Lactuca sativa treated with: (a) a commercial fish and seaweed hydrolysate; (b) acid hydrolysate; (c) base hydrolysate.

### DETAILED DESCRIPTION

Protein hydrolysates and methods for producing hydrolysates are provided herein. For example, protein hydrolysates may be produced from microbial biomass, as described herein.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the methods, systems, and compositions described herein.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

### I. Definitions

“A,” “an” and “the” include plural references unless the context clearly dictates, thus the indefinite articles “a”, “an,”, and “the” as used herein in the specification and in the claims, unless clearly indicated to the contrary, should be understood to mean “at least one.”

The term “about” as used herein when referring to a measurable value such as an amount, a temporal duration, and the like, is meant to encompass variations of ±5%, ±1%, or ±0.1% from the specified value, as such variations are appropriate to perform the disclosed methods or in connection with a disclosed composition.

“Acetogen” refers to a microorganism that generates acetate and/or other short chain organic acids up to C4 chain length as a product of anaerobic respiration.

“Acidophile” refers to a type of extremophile that thrives under highly acidic conditions (usually at pH 2.0 or below).

The term “amino acid” refers to a molecule containing both an amine group and a carboxyl group that are bound to a carbon, which is designated the alpha-carbon. Suitable amino acids include, without limitation, both the D- and L-isomers of the naturally occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “biomass” refers to a material produced by growth and/or propagation of cells. Biomass may contain cells and/or intracellular contents as well as extracellular material, including, but not limited to, compounds secreted by a cell.

The term “bioreactor” or “fermenter” refers to a closed or partially closed vessel in which cells are grown and maintained. The cells may be, but are not necessarily, held in liquid suspension. In some embodiments, rather than being held in liquid suspension, cells may alternatively be grown and/or maintained in contact with, on, or within another non-liquid substrate including but not limited to a solid growth support material.

“Bio-stimulants” or “Biostimulant” refers to compounds capable of stimulating the growth and development of plants, e.g., agricultural crops, as well as increasing and enhancing microbiological activity of the soil. The term also refers to any substance that may be applied to a plant, seed, soil, or growing media and which enhances a plant's nutrient use efficiency or provides other direct or indirect benefits to plant development or stress response.

The term “carbon fixing” process, reaction or pathway refers to enzymatic reactions or metabolic pathways that convert forms of carbon that are gaseous under ambient conditions, including but not limited to CO₂, CO, and CH₄, into carbon-based biochemicals that are liquid or solid under ambient conditions, or which are dissolved into, or held in suspension in, aqueous solution.

“Carbon source” refers to the types of molecules from which a microorganism derives the carbon needed for organic biosynthesis.

“Carboxydotrophic” refers to microorganisms that can tolerate or oxidize carbon monoxide. In preferred embodiments a carboxydotrophic microorganism can utilize CO as a carbon source and/or as a source of reducing electrons for biosynthesis and/or respiration.

“Chemoautotrophic” refers to organisms that obtain energy by the oxidation of chemical electron donors by chemical electron acceptors and synthesize all the organic compounds needed by the organism to live and grow from carbon dioxide.

In the claims, as well as in the specification, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

The term “culturing” refers to growing a population of cells, e.g., microbial cells, under suitable conditions for growth, in a liquid or solid medium.

The term “derived from” encompasses the terms “originated from,” “obtained from,” “obtainable from,” “isolated from,” and “created from,” and generally indicates that one specified material finds its origin in another specified material or has features that can be described with reference to another specified material.

“Energy source” refers to either the electron donor that is oxidized by oxygen in aerobic respiration or the combination of electron donor that is oxidized and electron acceptor that is reduced in anaerobic respiration.

“Extremophile” refers to a microorganism that thrives in physically or geochemically extreme conditions (e.g., high or low temperature, pH, or high salinity) compared to conditions on the surface of the Earth or the ocean that are typically tolerated by most life forms found on or near the earth's surface.

The term “gasification” refers to a generally high temperature process that converts carbon-based materials into a mixture of gases including hydrogen, carbon monoxide, and carbon dioxide called synthesis gas, syngas or producer gas. The process generally involves partial combustion and/or the application of externally generated heat along with the controlled addition of oxygen and/or steam such that insufficient oxygen is present for complete combustion of the carbon-based material.

“Halophile” refers to a type of extremophile that thrives in environments with very high concentrations of salt.

“Heterotrophic” refers to organisms that cannot synthesize all the organic compounds needed by the organism to live and grow from carbon dioxide, and which must utilize organic compounds for growth. Heterotrophic organisms cannot produce their own food and instead obtain food and energy by taking in and metabolizing organic substances, such as plant or animal matter, i.e., rather than fixing carbon from inorganic sources such as carbon dioxide.

“Hydrogen-oxidizer” refers to a microorganism that utilizes reduced H₂ as an electron donor for the production of intracellular reducing equivalents and/or in respiration.

“Hyperthermophile” refers to a type of extremophile that thrives in extremely hot environments for life, typically about 60° C. (140° F.) or higher.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP). The terms “oxyhydrogen” and “oxyhydrogen microorganism” can be used synonymously with “knallgas” and “knallgas microorganism,” respectively. Knallgas microorganisms generally use molecular hydrogen by means of hydrogenases, with some of the electrons donated from H₂ that is utilized for the reduction of NAD⁺ (and/or other intracellular reducing equivalents) and some of the electrons from H₂ that is used for aerobic respiration. Knallgas microorganisms generally fix CO₂ autotrophically, through pathways including but not limited to the Calvin Cycle or the reverse citric acid cycle [“Thermophilic bacteria”, Jakob Kristjansson, Chapter 5, Section III, CRC Press, (1992)].

The term “lysate” refers to the liquid containing a mixture and/or a solution of cell contents that result from cell lysis. In some embodiments, the methods described herein comprise a purification of chemicals or mixture of chemicals in a cellular lysate. In some embodiments, the methods comprise a purification of amino acids and/or protein in a cellular lysate.

The term “lysis” refers to the rupture of the plasma membrane and if present, the cell wall of a cell such that a significant amount of intracellular material escapes to the extracellular space. Lysis can be performed using electrochemical, mechanical, osmotic, thermal, or viral means. In some embodiments, the methods described herein comprise performing a lysis of cells or microorganisms as described herein in order to separate a chemical or mixture of chemicals from the contents of a bioreactor. In some embodiments, the methods comprise performing a lysis of cells or microorganisms described herein in order to separate an amino acid or mixture of amino acids and/or proteins from the contents of a bioreactor or cellular growth medium.

“Methanogen” refers to a microorganism that generates methane as a product of anaerobic respiration.

“Methylotroph” refers to a microorganism that can use reduced one-carbon compounds, such as but not limited to methanol or methane, as a carbon source and/or as an electron donor for their growth.

The terms “microorganism” and “microbe” mean microscopic single celled life forms.

“Mixotrophic” refers to an organism that is capable of utilizing a mixture of different energy sources and carbon, for example, H₂ and sugar.

The term “molecule” means any distinct or distinguishable structural unit of matter comprising one or more atoms, and includes for example hydrocarbons, lipids, polypeptides and polynucleotides.

“Oligopeptide” refers to a peptide that contains a relatively small number of amino-acid residues, for example, about 2 to about 20 amino acids.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

The term “organic compound” refers to any gaseous, liquid, or solid chemical compound that contains carbon atoms, with the following exceptions that are considered inorganic: carbides, carbonates, simple oxides of carbon, cyanides, and allotropes of pure carbon such as diamond and graphite.

“Peptide” refers to a compound consisting of two or more amino acids linked in a chain, the carboxyl group of each acid being joined to the amino group of the next by a bond of the type R—OC—NH—R′, for example, about 2 to about 50 amino acids.

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide,” “nucleic acid,” and “oligonucleotide” are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR.sub.2 (“amidate”), P(O)R, P(O)OR′, CO or CH.sub.2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, etc.), as well as other modifications known in the art.

The term “precursor to” or “precursor of” is an intermediate towards the production of one or more of the components of a finished product.

“Producer gas” refers to a gas mixture containing various proportions of H₂, CO, and CO₂, and having heat value typically ranging between one half and one tenth that of natural gas per unit volume under standard conditions. Producer gas can be generated various ways from a variety of feedstocks, including gasification, steam reforming, or autoreforming of carbon-based feedstocks. In addition to H₂, CO, and CO₂, producer gases can contain other constituents including but not limited to methane, hydrogen sulfide, condensable gases, tars, and ash depending upon the generation process and feedstock. The proportion of N₂ in the mixture can be high or low depending whether air is used as an oxidant in the reactor or not and if the heat for the reaction is provided by direct combustion or through indirect heat exchange.

The term “producing” includes both the production of compounds intracellularly and extracellularly, including the secretion of compounds from the cell.

“Psychrophile” refers to a type of extremophile capable of growth and reproduction in cold temperatures, typically about 10° C. and lower.

The terms “recovered,” “isolated,” “purified,” and “separated” as used herein refer to a material (e.g., a protein, nucleic acid, or cell) that is removed from at least one component with which it is naturally associated. For example, these terms may refer to a material that is substantially or essentially free from components which normally accompany it as found in its native state, such as, for example, an intact biological system.

The phrase “substantially free” as to any given component means that such component is only present, if at all, in an amount that is a functionally insignificant amount, i.e., it does not significantly negatively impact the intended performance or function of any process or product. Typically, substantially free means less than about 1%, including less than about 0.5%, including less than about 0.1%, and also including zero percent, by weight of such component.

“Sulfur-oxidizer” refers to microorganisms that utilize reduced sulfur containing compounds including but not limited to H₂S as electron donors for the production of intracellular reducing equivalents and/or in respiration.

“Syngas” or “Synthesis gas” refers to a type of gas mixture, which like producer gas contains H₂ and CO, but which has been more specifically tailored in terms of H₂ and CO content and ratio and levels of impurities for the synthesis of a particular type of chemical product, such as but not limited to methanol or Fischer-tropsch diesel. Syngas generally contains H₂, CO, and CO₂ as major components, and it can be generated through established methods including: steam reforming of methane, liquid petroleum gas, or biogas; or through gasification of any organic, flammable, carbon-based material, including but not limited to biomass, waste organic matter, various polymers, peat, and coal. The hydrogen component of syngas can be increased through the reaction of CO with steam in the water gas shift reaction, with a concomitant increase in CO₂ in the syngas mixture.

“Thermophile” refers to a type of extremophile that thrives at relatively high temperatures for life, typically about 45° C. to about 122° C.

“Wild-type” refers to a microorganism as it occurs in nature.

“Yield” refers to amount of a product produced from a feed material (for example, sugar) relative to the total amount of the substance that would be produced if all of the feed substance were converted to product. For example, amino acid yield may be expressed as % of amino acid produced relative to a theoretical yield if 100% of the feed substance were converted to amino acid.

### II. Methods

In general terms, a method of the present disclosure may include raising or lowering the pH of a proteinaceous suspension, e.g., a biomass suspension, for example, a suspension of microbial biomass, such as biomass produced by growth of a chemoautotrophic microorganism, thereby producing an alkaline or acidic suspension.

The starting biomass suspension may include a suitable amount of the biomass in the medium, for example, microbial biomass in a growth medium. In some embodiments, the amount of the biomass, dried weight/reaction volume (w/v), is about 0.1% or more, e.g., about 0.2% or more, about 0.5% or more, about 1% or more, about 2% or more, about 3% or more, including about 4% or more. In some embodiments, the amount of the biomass, dried weight/reaction volume, is about 8% or less, e.g., about 6% or more, including about 5% or less; and in some embodiments, each of the foregoing biomass ranges may be at least about 0.01%, at least about 0.2%, at least about 0.5%, at least about 1%, at least about 2%, or at least about 3%. In some embodiments, the amount of the biomass, dried weight/reaction volume, is in a range of about 0.1% to about 8%, e.g., about 0.2% to about 8%, about 0.5% to about 6%, about 1% to about 6%, about 2% to about 6%, including about 3% to about 5%.

In some embodiments, cells within the biomass are subjected to lysis at the beginning of the process, e.g., prior to raising or lowering the pH, to facilitate harvesting the protein from the biomass into a suspension composition.

The alkaline or acidic suspension is subjected to heat for a suitable amount of time, to generate a protein hydrolysate composition. The suspension may be concentrated, dried (e.g., lyophilized), or utilized directly as a liquid suspension. In certain embodiments, the alkaline or acidic suspension is subjected to heat and elevated pressure, e.g., by autoclaving the alkaline or acidic suspension, to generate a protein hydrolysate composition. In some embodiments, the suspension is neutralized with buffer to lower the pH after the heat or heat/pressure treatment.

In certain embodiments, the pH is lowered (for an alkaline suspension) or raised (for an acidic suspension) sufficiently to allow subsequent enzymatic treatment of the suspension with protease. After enzymatic hydrolysis, a protein hydrolysate composition is produced.

In certain embodiments, the hydrolyzed protein in the protein hydrolysate is predominantly in the soluble fraction of the suspension. The resulting suspension may be clarified, e.g., by centrifuge, to obtain a supernatant fraction, which contains hydrolyzed protein.

### A. Alkaline Hydrolysis

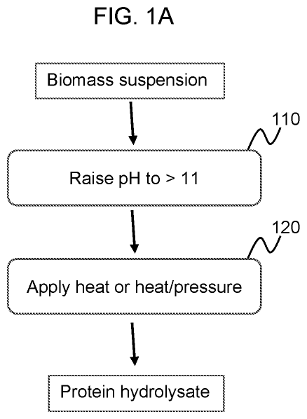

With reference to FIGS. 1A and 1B, a method of the present disclosure may include as the starting material a suspension of a cellular biomass. The suspension may be obtained by suspending (e.g., by homogenizing) a cellular biomass in a suitable liquid medium such as water or buffer or culture (growth) medium. Any suitable method of suspending a biomass in a medium may be used, including, but not limited to, vortexing, homogenizing, stirring, sonicating, etc. The biomass may be a dry biomass (e.g., lyophilized biomass) or wet biomass before suspending in the medium.

The pH of the biomass suspension may be increased 110, 130 by adding a base to the suspension. In some embodiments, the pH of the suspension is raised to about 10.0 or higher, e.g., about 11 or higher, about 11.1 or higher, about 11.2 or higher, about 11.3 or higher, about 11.4 or higher, about 11.5 or higher, about 11.6 or higher, about 11.7 or higher, about 11.8 or higher, about 11.9 of higher, including about 12.0 or higher. In some embodiments, the pH of the suspension is raised to about 10.0 to about 14.0, e.g., about 11.0 to about 14.0, about 10.0 to about 13.0, about 10.5 to about 13.0, about 10.5 to about 12.5, about 10.5 to about 11.5, about 11.0 to about 13.5, about 11.0 to about 13.0, about 11.0 to about 12.5, about 11.0 to about 12.0, about 11.5 to about 13.0, about 11.5 to about 12.5, about 11.5 to about 12.4, about 11.6 to about 12.4, about 11.7 to about 12.4, including about 11.8 to about 12.3. Any suitable base may be used to raise the pH of the suspension. A suitable base may include, but is not limited to, potassium hydroxide (KOH), ammonium hydroxide (NH₄OH), ammonia (NH₃), calcium hydroxide (Ca(OH)₂), and/or sodium hydroxide (NaOH). In certain embodiments, the base is KOH. In certain embodiments, one or more bases are used that contain plant nutrients, including, but not limited to, nitrogen (N), phosphorus (P), and/or potassium (K). In certain embodiments, base(s) are avoided that contain elements that inhibit plant growth, including, but not limited to, sodium (Na).

The alkaline biomass suspension may be subjected to elevated temperature 120, 140. In some embodiments, the suspension is subjected to a temperature of about 40° C. or higher, e.g., about 50° C. or higher, about 60° C. or higher, about 70° C. or higher, about 80° C. or higher, about 90° C. or higher, about 100° C. or higher, about 105° C. or higher, about 110° C. or higher, including about 121° C. or higher. In some embodiments, the suspension is subjected to a temperature of about 40° C. to about 150° C., e.g., about 60° C. to about 150° C., about 70° C. to about 140° C., about 80° C. to about 140° C., about 90° C. to about 135° C., about 100° C. to about 130° C., about 100° C. to about 125° C., about 105° C. to about 125° C., including about 110° C. to about 125° C. In some embodiments, the suspension is subjected to a temperature of about 110° C. The suspension may be subjected to elevated temperature using any suitable method.

In some embodiments, the alkaline biomass suspension is subjected to elevated pressure 120, 140 (i.e., at least 14.7 psi). In some embodiments, the suspension is subjected to a pressure of about 15 psi or higher, e.g., about 20 psi or higher, about 25 psi or higher, about 30 psi or higher, about 35 psi or higher, or about 40 psi or higher, and in some embodiments, an a pressure of about 40 psi or lower, e.g., about 30 psi or lower, about 25 psi or lower, about 20 psi or lower, about 18 psi or lower, including about 15 psi or lower. In some embodiments, the suspension is subjected to a pressure in the range of about 14.7 psi to about 40 psi, e.g., about 15 psi to about 40 psi, about 15 psi to about 35 psi, about 15 psi to about 30 psi, about 15 psi to about 25 psi, or about 15 psi to about 20 psi. The suspension may be subjected to elevated temperature and pressure using any suitable method. In some embodiments, the suspension is autoclaved to raise the temperature and pressure. In some embodiments, the suspension is exposed to an elevated pressure for only a portion of the time that it is exposed to the elevated temperature. In some embodiments, the suspension is exposed to an elevated pressure for at least about 50%, at least about 55%, at least about 60%, at least about 65%, at least about 70%, at least about 75%, at least about 80%, at least about 85%, at least about 90%, or at least about 95% of the time that it is exposed to the elevated temperature.

The alkaline biomass suspension may be subjected to elevated temperature, or elevated temperature and pressure, for a suitable period of time. In some embodiments, the suspension is subjected to elevated temperature, or elevated temperature and pressure, for about 5 minutes or more, e.g., about 10 minutes or more, about 15 minutes or more, about 20 minutes or more, including about 25 minutes or more, and in some embodiments each of the foregoing time periods may be capped at about 90 minutes, about 80 minutes, about 70 minutes, about 60 minutes, about 50 minutes, about 40 minutes, or about 30 minutes. In some embodiments, the suspension is subjected to elevated pressure and temperature for a time period from about 5 minutes to about 90 minutes, e.g., from about 5 minutes to about 80 minutes, from about 5 minutes to 70 minutes, from about 10 minutes to about 60 minutes, from about 10 minutes to about 50 minutes, from about 10 to about 40 minutes, from about 10 to about 30 minutes, from about 20 to about 60 minutes, including from about 30 to about 60 minutes. In some embodiments, the suspension is subjected to elevated temperature, or elevated temperature and pressure, for about 1 hour or more, e.g., about 3 hours or more, about 5 hours or more, including about 8 hours or more, and in some embodiments each of the foregoing time periods may be capped at about 24 hours, about 18 hours, about 12 hours, or about 8 hours. In some embodiments, the suspension is subjected to elevated temperature, or elevated temperature and pressure, for about 1 hour to about 24 hours, e.g., about 1 hour to about 18 hours, about 1 hour to about 12 hours, including about 1 hour to about 8 hours.

In some embodiments, after the temperature and heat treatment, the pH of the suspension is reduced 150 by adding a neutralizing agent, e.g., a neutralizing buffer. In some embodiments, the pH is reduced to about 6.5 or higher, e.g., about 6.7 or higher, about 6.9 or higher, about 7.0 or higher, about 7.1 or higher, about 7.2 or higher, about 7.3 or higher, about 7.4 or higher, about 7.5 or higher, about 7.6 or higher, about 7.7 or higher, about 7.8 or higher, about 7.9 or higher, including about 8.0 or higher; and in some embodiments each of the foregoing pH ranges may be capped at 8.5, 8.6, 8.7, 8.8, 8.9, 9.0, 9.1, 9.2, 9.3, 9.4, or 9.5. In some embodiments, the pH is reduced to about 6.5 to about 9.5, e.g., about 7.0 to about 9.5, about 7.3 to about 9.4, about 7.5 to about 9.4, about 7.7 to about 9.3, about 7.8 to about 9.2, about 7.9 to about 9.1, about 8.0 to about 9.0, including about 8.5 to about 9.0. The pH may be lowered using any suitable neutralizing agent. A suitable neutralizing agent includes, but is not limited to, a potassium phosphate buffer, ammonium phosphate buffer, sodium citrate buffer, sodium phosphate buffer, Tris buffer, HEPES buffer, and/or glycine buffer. In some embodiments, the neutralizing agent is a potassium phosphate buffer. In some embodiments, the neutralizing agent includes, but is not limited to, ammonium phosphate, sodium citrate, potassium citrate, citric acid, sodium phosphate, potassium phosphate, or phosphoric acid. In some embodiments, the pH is lowered using CO₂ and/or bicarbonate and/or carbonic acid. In certain embodiments where ammonia, ammonium hydroxide, calcium hydroxide, or calcium oxide is used as a base, CO₂ is used for neutralization. In certain embodiments where calcium hydroxide, or calcium oxide is used as a base, phosphoric acid or phosphate buffer is used for neutralization. In some embodiments, the pH is lowered using an organic acid. In certain embodiments, one or more neutralizing agents or buffers are used that contain plant nutrients, including but not limited to nitrogen (N), phosphorus (P), and/or potassium (K). In certain embodiments, the neutralizing agents or buffers do not contain elements that are inhibitory to plant growth, including but not limited to sodium (Na) and/or chloride (Cl). In certain embodiments, a neutralizing agent or buffer is not added following the heat/pressure step.

After the alkaline hydrolysis, proteins in the composition are broken down into polypeptides of varying sizes. In accordance with an embodiment, after alkaline hydrolysis, at least 60% or at least 65% or at least 70% or at least 75% or at least 80% or at least 85% or at least 90% or at least 95% of the polypeptides in the suspension, e.g., the soluble fraction of the suspension, have an atomic mass of less than about 30 kD, less than about 25 kD, less than about 20 kD, less than about 15 kD, less than about 10 kD, less than about 5 kD, less than about 3 kD, or less than about 2 kD; and in some embodiments, each of the foregoing mass ranges may be at least about 0.1 kD, at least about 0.5 kD, at least about 1 kD, or at least about 5 kD. In accordance with an embodiment, at least 60% or at least 65% or at least 70% or at least 75% or at least 80% or at least 85% or at least 90% or at least 95% of the polypeptides in the suspension, e.g., the soluble fraction of the suspension, have an atomic mass of from about 0.1 kD to about 30 kD, 1 kD to about 30 kD, from about 1 kD to about 25 kD, from about 5 kD to about 25 kD, from about 0.1 kD to about 10 kD, from about 0.1 kD to about 5 kD, from about 0.1 kD to about 2 kD, or from about 5 kD to about 20 kD.

### B. Acid Hydrolysis

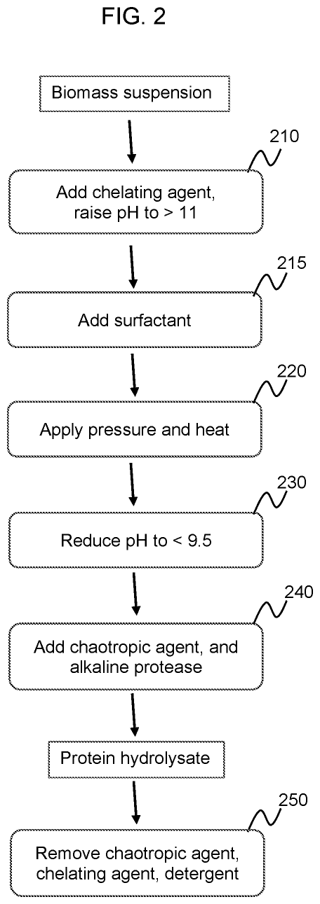

With reference to FIGS. 3A and 3B, a method of the present disclosure may include as the starting material a suspension of a cellular biomass. The suspension may be obtained by suspending (e.g., by homogenizing) a cellular biomass in a suitable liquid medium such as water or buffer or culture (growth) medium. Any suitable method of suspending a biomass in a medium may be used, including, but not limited to, vortexing, homogenizing, stirring, sonicating, etc. The biomass may be a dry biomass (e.g., lyophilized biomass) or wet biomass before suspending in the medium.

The pH of the biomass suspension may be increased 310, 330 by adding an acid to the suspension. In some embodiments, the pH of the suspension is lowered to about 0.5 to about 3. Any suitable acid may be used to lower the pH of the suspension. A suitable acid may include, but is not limited to, one or more of phosphoric acid (H₃PO₄), sulfuric acid (H₂SO₄), nitric acid (HNO₃), formic acid (HCOOH), acetic acid (CH₃COOH), carbonic acid i.e. carbon dioxide (CO₂) and hydrochloric acid (HCl). In certain embodiments, one or more acids are used that contain plant nutrients, including, but not limited to, nitrogen (N), phosphorus (P), and/or potassium (K). In certain embodiments, acid(s) are avoided that contain elements that inhibit plant growth, including, but not limited to, chloride (Cl).

The acidic biomass suspension may be subjected to elevated temperature 320, 340. In some embodiments, the suspension is subjected to a temperature of about 40° C. or higher, e.g., about 50° C. or higher, about 60° C. or higher, about 70° C. or higher, about 80° C. or higher, about 90° C. or higher, about 100° C. or higher, about 105° C. or higher, about 110° C. or higher, including about 121° C. or higher. In some embodiments, the suspension is subjected to a temperature of about 40° C. to about 150° C., e.g., about 60° C. to about 150° C., about 70° C. to about 140° C., about 80° C. to about 140° C., about 90° C. to about 135° C., about 100° C. to about 130° C., about 100° C. to about 125° C., about 105° C. to about 125° C., including about 110° C. to about 125° C. In some embodiments, the suspension is subjected to a temperature of about 110° C. The suspension may be subjected to elevated temperature using any suitable method.

In some embodiments, the acidic biomass suspension is subjected to elevated pressure 320, 340 (i.e., at least 14.7 psi). In some embodiments, the suspension is subjected to a pressure of about 15 psi or higher, e.g., about 20 psi or higher, about 25 psi or higher, about 30 psi or higher, about 35 psi or higher, or about 40 psi or higher, and in some embodiments, a pressure of about 40 psi or lower, e.g., about 30 psi or lower, about 25 psi or lower, about 20 psi or lower, about 18 psi or lower, including about 15 psi or lower. In some embodiments, the suspension is subjected to a pressure in the range of about 14.7 psi to about 40 psi, e.g., about 15 psi to about 40 psi, about 15 psi to about 35 psi, about 15 psi to about 30 psi, about 15 psi to about 25 psi, or about 15 psi to about 20 psi. The suspension may be subjected to elevated temperature and pressure using any suitable method. In some embodiments, the suspension is autoclaved to raise the temperature and pressure. In some embodiments, the suspension is exposed to an elevated pressure for only a portion of the time that it is exposed to the elevated temperature. In some embodiments, the suspension is exposed to an elevated pressure for at least about 50%, at least about 55%, at least about 60%, at least about 65%, at least about 70%, at least about 75%, at least about 80%, at least about 85%, at least about 90%, or at least about 95% of the time that it is exposed to the elevated temperature.

The acidic biomass suspension may be subjected to elevated temperature, or elevated temperature and pressure, for a suitable period of time. In some embodiments, the suspension is subjected to elevated temperature, or elevated temperature and pressure, for about 5 minutes or more, e.g., about 10 minutes or more, about 15 minutes or more, about 20 minutes or more, including about 25 minutes or more, and in some embodiments each of the foregoing time periods may be capped at about 90 minutes, about 80 minutes, about 70 minutes, about 60 minutes, about 50 minutes, about 40 minutes, or about 30 minutes. In some embodiments, the suspension is subjected to elevated pressure and temperature for a time period from about 5 minutes to about 90 minutes, e.g., from about 5 minutes to about 80 minutes, from about 5 minutes to 70 minutes, from about 10 minutes to about 60 minutes, from about 10 minutes to about 50 minutes, from about 10 to about 40 minutes, from about 10 to about 30 minutes, from about 20 to about 60 minutes, including from about 30 to about 60 minutes. In some embodiments, the suspension is subjected to elevated temperature, or elevated temperature and pressure, for about 1 hour or more, e.g., about 3 hours or more, about 5 hours or more, including about 8 hours or more, and in some embodiments each of the foregoing time periods may be capped at about 24 hours, about 18 hours, about 12 hours, or about 8 hours. In some embodiments, the suspension is subjected to elevated temperature, or elevated temperature and pressure, for about 1 hour to about 24 hours, e.g., about 1 hour to about 18 hours, about 1 hour to about 12 hours, including about 1 hour to about 8 hours.

In some embodiments, after the temperature and heat treatment, the pH of the suspension is increased 350 by adding a neutralizing agent, e.g., a neutralizing buffer. In some embodiments, the pH is increased to about 6 or to about 7. The pH may be raised using any suitable neutralizing agent. A suitable neutralizing agent includes, but is not limited to, bicarbonate buffer or phosphate buffer. In some embodiments, the neutralizing agent includes, but is not limited to, calcium hydroxide (Ca(OH)₂)), calcium carbonate (CaCO₃), potassium hydroxide (KOH), or ammonium hydroxide (NH₄OH). In certain embodiments, one or more neutralizing agents or buffers are used that contain plant nutrients, including but not limited to nitrogen (N), phosphorus (P), and/or potassium (K). In certain embodiments, the neutralizing agents or buffers do not contain elements that are inhibitory to plant growth, including but not limited to sodium (Na) and/or chloride (Cl). In certain embodiments, a neutralizing agent or buffer is not added following the heat/pressure step.

After the acidic hydrolysis, proteins in the composition are broken down into polypeptides of varying sizes. In accordance with an embodiment, after acid hydrolysis, at least 60% or at least 65% or at least 70% or at least 75% or at least 80% or at least 85% or at least 90% or at least 95% of the polypeptides in the suspension, e.g., the soluble fraction of the suspension, have an atomic mass of less than about 30 kD, less than about 25 kD, less than about 20 kD, less than about 15 kD, less than about 10 kD, less than about 5 kD, less than about 3 kD, or less than about 2 kD; and in some embodiments, each of the foregoing mass ranges may be at least about 0.1 kD, at least about 0.5 kD, at least about 1 kD, or at least about 5 kD. In accordance with an embodiment, at least 60% or at least 65% or at least 70% or at least 75% or at least 80% or at least 85% or at least 90% or at least 95% of the polypeptides in the suspension, e.g., the soluble fraction of the suspension, have an atomic mass of from about 0.1 kD to about 30 kD, from about 1 kD to about 30 kD, from about 1 kD to about 25 kD, from about 5 kD to about 25 kD, from about 0.1 kD to about 2 kD, from about 0.1 kD to about 5 kD, from about 0.1 kD to about 10 kD, or from about 5 kD to about 20 kD.

### C. Protease Treatment

In some embodiments, one or more protease may be added 160, 360 to the neutralized suspension and incubated under appropriate conditions for additional hydrolysis of the biomass, for example, in embodiments in which alkaline or acid hydrolysis, as described above, is incomplete or does not result in peptides with a desired peptide size range and/or properties. The protease may be any suitable protease. The protease may be an endoproteinase and/or an exoproteinase. In some embodiments, the protease is an alkaline protease. In some embodiments, the alkaline protease is a serine alkaline protease. In some embodiments, the alkaline protease is a bacterial alkaline protease. In some embodiments, the alkaline protease includes, without limitation, subtilisin A. In some embodiments, the protease is an acid protease, such as an aspartic or glutamic protease. In some embodiments, the protease is a metalloprotease. In certain embodiments, combinations of one or more types of proteolytic enzymes are utilized, such as exoprotease, endoprotease, neutral protease, carboxypeptidase, and/or aminopeptidase. In certain such embodiments, one or more of the exoprotease, endoprotease, neutral protease, carboxypeptidase, and aminopeptidase is derived from Aspergillus oryzae. In certain embodiments, an endoprotease enzyme preparation derived from Bacillus subtilis is utilized. In certain embodiments, a glutaminase is utilized. In certain such embodiments, the glutaminase is derived from Aspergillus niger.

In certain non-limiting embodiments, the microorganisms are hydrolyzed with at least one enzyme that is capable of hydrolyzing microbial (e.g., bacterial) proteins into free amino acids and/or short peptides. In certain embodiments, enzymatic hydrolysis comprises hydrolyzing with a purified enzyme. In certain embodiments, enzymatic hydrolysis comprises hydrolyzing with a mixture of an enzyme and a medium in which the enzyme was prepared. In certain embodiments, enzymatic hydrolysis comprises hydrolyzing with an enzyme of plant and/or animal and/or bacterial and/or archaea and/or fungal origin. In certain embodiments, enzymatic hydrolysis comprises hydrolyzing with a mixture of one or more enzyme(s) of plant, animal, bacterial, archaea, and/or fungal origin. In certain embodiments, the hydrolytic enzyme is produced by a microorganism strain as described herein. In certain embodiments, the hydrolytic enzyme is produced from microorganisms grown on Cl substrates and/or H₂ and/or syngas feedstock. In certain embodiments, bacterial cells may be hydrolyzed with one or more of proteases, lipases and amylases. In certain embodiments, enzymatic hydrolysis comprises one or more proteolytic enzyme(s) of microbial, plant, fungal, and/or animal origin. In certain embodiments, the method includes use of an alkaline protease. In certain embodiments, enzymatic hydrolysis comprises hydrolyzing with at least one enzyme selected from pancreatin, papain, bromelain, ficin, bacterial protease, fungal protease, a neutral protease produced by Bacillus sp. Alcalase 2.4 L, Bacillus B. licheniformis, and/or Subtilisin carlesberg, Esperase from B. lentus, Nutrase from B. amyloliquifacus, Protamex from Bacillus sp., Therolysin/therolase from B. thermoproteolyticus, Flavouzyme from Aspergillus oryzae, Protease N from B. subtilis, trypsin, chymotrypsin, keratinase, pepsin, subtilisin, and/or rennin. As would be well understood by one of ordinary skill in the art, pancreatin includes a mixture of digestive enzymes, proteases, lipases and amylases.

Enzymatically hydrolyzing microbial (e.g., bacterial) cells may include combining an enzyme and microbial (e.g., bacterial) cells in any suitable amount under any suitable conditions. In certain embodiments, quantities of reactants, reaction conditions, and sequences of reaction steps are selected to achieve ideal enzyme activity. In certain embodiments the conditions of pressure, temperature, pH and time of the enzymatic hydrolysis are those in which maximum or a suitable level of enzyme activity is achieved. In certain embodiments performing enzymatic hydrolysis comprises combining an enzyme and the microbial (e.g., bacterial) cells in a weight ratio ranging from about 0.1 to about 10 g of enzyme per 100 g of nitrogen content of microbial (e.g., bacterial) cells. In another particular embodiment, the enzymatic hydrolysis is performed using a concentration of about 0.05% to about 0.5% by volume of enzyme stock solution with an activity of about 70,000 units, e.g., using the azocasein assay. In various embodiments, any suitable method may be employed to improve the efficiency of the enzymatic hydrolysis of the microbial (e.g., bacterial) cells. In certain embodiments, enzymatic hydrolysis comprises combining an enzyme and the microbial cells and agitating the combined enzyme and microbial cells by any suitable method. The enzymatic treatment can be performed in any suitable device known to one skilled in the art, such as a reactor with temperature control and stirring, for example.

The suspension may be incubated with the protease at a suitable pH and temperature for suitable or optimal catalytic activity of the specific protease that is utilized, and for a suitable amount of time to achieve the desired amount of proteolysis. In one embodiment, the suspension is incubated at about 55° C., for a suitable amount of time, e.g., about 3 hours or more, about 6 hours or more, about 12 hours or more, about 18 hours or more, or about 24 hours or more. In some embodiments, the suspension is incubated with a bacterial alkaline protease at about 55° C. overnight.

In certain embodiments, following incubation with a protease, the protease is inactivated. In certain embodiments, the protease is inactivated by a heat treatment. For example, the heat treatment may comprise raising the temperature to about 95° C. for about ten minutes.

Protease hydrolysis results in breakdown of proteins in the composition into polypeptides of varying sizes. In accordance with an embodiment, after proteolysis, at least 60% or at least 65% or at least 70% or at least 75% or at least 80% or at least 85% or at least 90% or at least 95% of the polypeptides in the suspension, e.g., the soluble fraction of the suspension, have an atomic mass of less than about 30 kD, less than about 25 kD, less than about 20 kD, less than about 15 kD, less than about 10 kD, less than about 5 kD, less than about 3 kD, or less than about 2 kD; and in some embodiments, each of the foregoing mass ranges may be at least about 0.1 kD, at least about 0.5 kD, at least about 1 kD, or at least about 5 kD. In accordance with an embodiment, at least 60%, at least 65%, at least 70%, at least 75%, at least 80%, at least 85%, at least 90%, or at least 95% of the polypeptides in the suspension, e.g., the soluble fraction of the suspension, have an atomic mass of from about 0.1 kD to about 30 kD, from about 1 kD to about 30 kD, from about 1 kD to about 25 kD, from about 5 kD to about 25 kD, from about 0.1 kD to about 2 kD, from about 0.1 kD to about 5 kD, from about 0.1 kD to about 10 kD, or from about 5 kD to about 20 kD.

### D. Pre-Treatment

Prior to the protein hydrolysis step or steps, in certain embodiments the biomass is subjected to one or more pre-treatment steps, such as but not limited to, cell lysis and/or defatting, i.e., lipid extraction. In certain embodiments defatting is performed using one or more solvent, including but not limited to, methanol, ethanol, isopropyl alcohol, hexane, acetone, propylene carbonate, dichloromethane, and/or chloroform. In certain embodiments, defatting is performed using one or more base, including but not limited to, ammonium hydroxide, ammonia, sodium hydroxide, and/or potassium hydroxide. In certain embodiments, defatted biomass is subjected to one or more of the protein hydrolysis methods described herein. In certain embodiments, a defatted biomass is subjected to enzymatic hydrolysis, in some embodiments with a relatively small pH adjustment required. In certain such embodiments, the pH is only adjusted to a pH of about 9 to about 5, at which pH enzymatic hydrolysis is performed. In certain embodiments, enzymatic hydrolysis is performed on the defatted biomass with no pH adjustment following the completion of the defatting step.

### E. Clarification/Separation

In some embodiments, the hydrolytic treatment (e.g., alkaline or acid hydrolysis, optionally including protease treatment) is followed by clarification of the suspension (hydrolysate) to remove undissolved material in the suspension, e.g., separation of soluble and insoluble fractions. The suspension may be clarified using any suitable method, such as centrifugation, filtration, etc. In some embodiments, after the suspension is clarified, e.g., centrifuged, the supernatant may be separated from the pellet.

In some embodiments, the clarified liquid composition (e.g., soluble fraction, such as supernatant of separated suspension), which contains hydrolyzed protein, is dried, e.g., lyophilized to produce a dry or substantially dry composition. In some embodiments, the lyophilized composition has a water content of about 10% or less, e.g., about 8% or less, about 6% or less, about 5% or less, including about 3% or less. In some embodiments, the lyophilized protein hydrolysate composition has a water content from about 1% to about 10%, e.g., about 1% to about 8%, about 1% to about 6%, including about 2% to about 5%.

In some embodiments, the clarified liquid composition (e.g., soluble fraction, such as supernatant of separated suspension) is dewatered or concentrated to lower the water content. In some embodiments, the concentrated composition has a water content of about 80% or less, e.g., about 75% or less, about 50% or less, about 40% or less, including about 30% or less; and in some embodiments, each of the foregoing water content ranges may be at least about 20%, at least about 25%, at least about 30%, at least about 40%, or at least about 50% (to the extent such foregoing ranges exceed such lower limits). In some embodiments, the dewatered product is dried using heat and/or evaporation, employing a method such as, but not limited to, one or more of spray drying; drum drying; oven drying; vacuum drying; vacuum oven drying; drying under an inert gas such as N₂; and solar evaporation. In some embodiments, the clarified product is dewatered initially with a rotary evaporator, e.g., such that about 50% to about 65% of the moisture is removed. In some embodiments, further dewatering is achieved by lyophilization, e.g., such that the lyophilized protein hydrolysate composition has a water content from about 1% to about 10%, e.g., about 1% to about 8%, about 1% to about 6%, including about 2% to about 5%.

In some embodiments, methods of the present disclosure include processing the insoluble fraction of the suspension, e.g., after alkaline or acid hydrolysis or after protease (e.g., alkaline protease, acid protease, or metalloprotease) treatment, to obtain a co-product. In some embodiments, the treated suspension is separated into soluble and insoluble fractions, e.g., centrifuged, to generate a supernatant fraction, containing a protein hydrolysate composition, and a pellet fraction. The pellet fraction may be processed further to extract a co-product. The co-product may be a function of the microorganism from which the biomass is obtained. In some embodiments, the co-product is a biopolymer, such as, a polyhydroxyalkanoate (PHA). The polyhydroxyalkanoate may include, without limitation, polyhydroxybutyrate (PHB).

### F. Treatment with Additional Agents

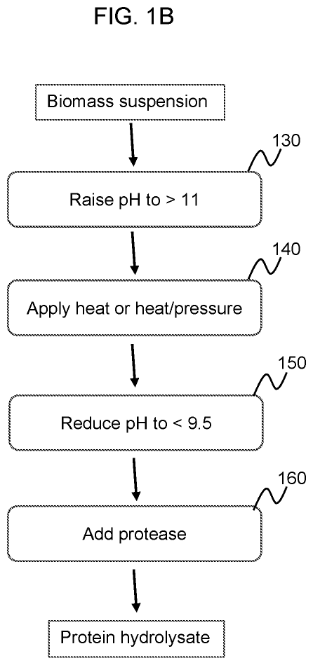

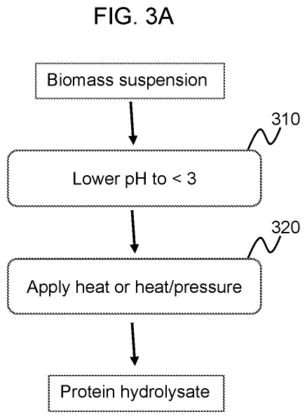

A chelating agent may be included in any of the embodiments of the biomass hydrolysis methods disclosed herein, e.g., for removal of undesired metal ions. A chelating agent may be added to the biomass suspension before subjecting the suspension to elevated heat or heat/pressure (see, e.g., FIGS. 2 and 4 (210, 410)). Any suitable chelating agent may be used, including natural chelating agents (e.g., amino acids) and synthetic chelating agents. Suitable chelating agents include, without limitation, ethylenediaminetetraacetic acid (EDTA) and ethylene glycol-bis(β-aminoethyl ether)-N,N,N′,N′-tetraacetic acid (EGTA). Any suitable amount of the chelating agent may be added to the biomass suspension, e.g., an amount that is sufficient to sequester undesired metal ions in the suspension. In some embodiments, the amount of chelating agent in the biomass suspension is in the range of about 0.1 mM to about 10 mM, e.g., about 0.5 mM to about 8 mM, about 1 mM to about 7 mM, including about 3 mM to about 5 mM. In some embodiments, the amount of chelating agent in the biomass suspension is about 5 mM. In certain embodiments, a chelating agent is not added to the biomass suspension before subjecting the suspension to elevated heat or heat/pressure. In certain embodiments, a synthetic chelating agent is not added to the biomass suspension before subjecting the suspension to elevated heat or heat/pressure. In certain embodiments, one or more chelating agents are used that contain one or more plant nutrients, including, but not limited to, nitrogen (N), phosphorus (P), and/or potassium (K). In certain embodiments, the chelating agents do not contain substances that are inhibitory to plant growth, including, but not limited to, sodium (Na) and/or chloride (Cl).

A surfactant may be included in any of the embodiments of the biomass hydrolysis methods disclosed herein, e.g., to improve solubility of the suspension. In some embodiments, a surfactant is added to the biomass suspension before subjecting the suspension to elevated heat or heat/pressure (see, e.g., FIGS. 2 and 4 (210, 410)). Any suitable surfactant may be added to the biomass suspension, including natural surfactants (i.e., taken directly from a natural source) or synthetic surfactants. Suitable surfactants include, but are not limited to, sodium dodecyl sulfate (SDS), ammonium lauryl sulfate, Triton X-100, Tween 80, Tween 20, and Pluronic PF-68. Any suitable amount of surfactant may be added to the biomass suspension. In some embodiments, the amount of surfactant in the biomass suspension is in the range of about 1% to about 25%, e.g., about 2% to about 20% about 4% to about 15%, about 5% to about 12%, including about 8% to about 12%, relative to the dry weight of biomass in the suspension (w/w). In some embodiments, the amount of surfactant added to the biomass suspension is about 10% (w/w), relative to the dry weight of biomass in the suspension. In certain embodiments, a surfactant is not added to the biomass suspension before subjecting the suspension to elevated heat or heat/pressure. In certain embodiments, a synthetic surfactant is not added to the biomass suspension before subjecting the suspension to elevated heat or heat/pressure. In certain embodiments, one or more surfactants are used that contain one or more plant nutrients, including, but not limited to, nitrogen (N), phosphorus (P), and/or potassium (K). In certain embodiments, the inclusion of surfactants contain substances that are inhibitory to plant growth is avoided, including, but not limited to, sodium (Na) and/or chloride (Cl). In certain embodiments, the added surfactant(s) are not toxic to plants or animals, and/or are biodegradable, and/or do not have detrimental environmental impacts.

A chaotropic agent may be included in any of the embodiments of the biomass hydrolysis methods disclosed herein, e.g., to aid in denaturing or unfolding of protein molecules. In some embodiments, a chaotropic agent is added before subjecting the suspension to elevated heat or heat/pressure. In some embodiments, a chaotropic agent is added (see, e.g., FIGS. 2 and 4 (240, 440)) to the neutralized suspension, including natural or synthetic chaotropic agents. In some embodiments, protease hydrolysis is carried out in the presence of a chaotropic agent in the neutralized suspension. Any suitable chaotropic agent may be added to the neutralized suspension. Suitable chaotropic agents include, without limitation, urea, thiourea and guanidium chloride. The chaotropic agent may be added to the neutralized suspension in any suitable amount. In some embodiments, the amount of chaotropic agent in the neutralized suspension is in the range of about 0.1 M to about 2 M, e.g., about 0.5 M to about 1.5 M, including about 0.8 M to about 1.2 M. In some embodiments, the amount of chaotropic agent in the neutralized suspension is about 1 M. In certain embodiments, a chaotropic agent is not added to the biomass suspension. In certain embodiments, a synthetic chaotropic agent is not added to the biomass suspension. In certain embodiments, one or more chaotropic agents are used that contain plant nutrients, including, but not limited to nitrogen (N), phosphorus (P), and/or potassium (K). In certain embodiments, the chaotropic agent does not contain substances that are inhibitory to plant growth, including, but not limited to, sodium (Na) and/or chloride (Cl).

In some embodiments, a method of the present disclosure includes removing a surfactant, chelating agent and/or chaotropic agent. In some embodiments, the surfactant, chelating agent, and/or chaotropic agent is removed after the treatment with a protease (e.g., an alkaline protease, acid protease, or metalloprotease) (see, e.g., FIGS. 2 and 4 (250, 450)) or in any other embodiment described herein that does not include protease treatment, e.g., via buffer exchange. Any suitable method(s) may be used to remove the surfactant, chelating agent and/or chaotropic agent, including, but not limited to, gel filtration chromatography, membrane filtration, and/or dialysis. In some embodiments, a protein hydrolysate composition of the present disclosure is produced without using a surfactant, chelating agent or chaotropic agent.

### III. Sources of Biomass

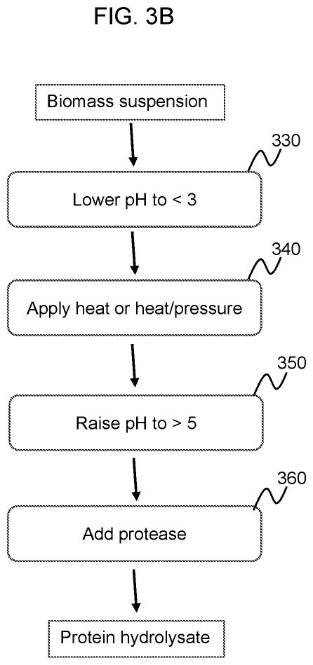

The methods of the present disclosure may be used to process a biomass suspension derived from any suitable source of proteinaceous material. The organism from which the protein hydrolysate composition is derived may be multicellular or single celled. In some embodiments, the protein hydrolysate composition of the present disclosure has a microbial origin. The microbial organism from which the protein hydrolysate composition is derived may be a photoautotrophic, heterotrophic, methanotrophic, methylotrophic, carboxydotrophic or chemoautotrophic organism. The microbial organism may be a wild-type, or it may be genetically modified. With reference to FIG. 5, the biomass may be collected from a culture of a suitable microorganism, e.g., in a fermenter or bioreactor 510. A biomass may be collected using any suitable method, such as a centrifuge, to separate the cell mass from the culture medium. The collected biomass may be used in the methods of the present disclosure 520 to produce a protein hydrolysate composition 530. In some embodiments, the collected biomass is spray dried or lyophilized to generate a dry biomass, which then may be used to produce a protein hydrolysate composition according to methods of the present disclosure.

In some embodiments, the microorganism is chosen from the genera Rhodococcus or Gordonia. In some embodiments, the microorganism is Rhodococcus opacus. In some embodiments, the microorganism is Rhodococcus opacus (DSM 43205) or Rhodococcus sp. (DSM 3346). In some embodiments, the microorganism is chosen from the genera Ralstonia or Cupriavidus or Hydrogenobacter. In some embodiments, the microorganism is Cupriavidus necator or Cupriavidus metallidurans. In some embodiments, the microorganism is Rhodococcus opacus; Hydrogenovibrio marinus; Rhodopseudomonas capsulata; Hydrogenobacter thermophilus; and Rhodobacter sphaeroides. In some non-limiting embodiments, the strain of Cupriavidus necator is DSM 531 or DSM 541. In some embodiments, the microorganism is a strain within the family burkholderiaceae. In some embodiments, the microorganisms is a strain within the genus Cupriavidus or Ralstonia. In some embodiments, the microorganism includes the species Cupriavidus necator. In some embodiments, the microorganism is a strain of the species Cupriavidus necator DSM 531. In some embodiments, the microorganism includes the species Cupriavidus metallidurans. In some embodiments, the microorganism is a strain of the species Cupriavidus metallidurans DSM 2839. In some embodiments, the microorganism includes the species Xanthobacter autotrophicus. In some embodiments, the microorganism is a strain of the species Xanthobacter autotrophicus DSM 432.

In some embodiments, a consortium of microorganisms is used as a source of biomass in the methods described herein. The consortium may include one or more of any of the microorganism species or strains or microorganisms having one or more microorganism traits described herein.

In some embodiments, a microorganism as described herein can accumulate protein to about 50% or more of the total cell mass by weight. In some embodiments, a microorganism as described herein can accumulate protein to about 60% or more of the total cell mass by weight. In some embodiments, the microorganism can accumulate protein to about 70% or more of the total cell mass by weight. In some embodiments, the microorganism can accumulate protein to about 80% or more of the total cell mass by weight. In some non-limiting embodiments, the microorganism exhibiting these traits is Cupriavidus necator DSM 531 or 541.

In some embodiments, a microorganism as described herein can naturally grow on H₂/CO₂ and/or syngas, and the microorganism can naturally accumulate polyhydroxybutyrate (PHB) or polyhydroxyalkanoate (PHA) to about 50% or more of the cell biomass by weight. In some embodiments, the microorganism has a native ability to direct a high flux of carbon through the acetyl-CoA metabolic intermediate, which can lead into fatty acid biosynthesis, along with a number of other synthetic pathways, for example, PHA, e.g., PHB synthesis, and/or amino acid biosynthesis. In some embodiments, the microorganism exhibiting these traits is Cupriavidus necator (e.g., DSM 531 or DSM 541).

In some embodiments, a wild-type microorganism is able produce a PHA, such as PHB, and a mutant or engineered strain of the microorganism that produces less PHA (e.g., PHB) than the wild-type strain, when grown under the same conditions, is used in a method described herein. In some embodiments the mutant or engineered strain is not able to produce detectable amounts of PHA (e.g., PHB). In certain embodiments, a (PHA (e.g., PHB)-negative mutant is used. In certain such embodiments, the species is Cupriavidus necator. In certain such embodiments, the strain is Cupriavidus necator DSM 541.

In some nonlimiting embodiments, the microorganism is Corynebacterium autotrophicum. In some nonlimiting embodiments, the microorganism is Corynebacterium autotrophicum and/or Corynebacterium glutamicum. In some embodiments, the microorganism is Hydrogenovibrio marinus. In some embodiments, the microorganism is Rhodopseudomonas capsulata, Rhodopseudomonas palustris, or Rhodobacter sphaeroides.

In some embodiments, the microorganism cells comprise microorganisms selected from one or more of the following genera: Cupriavidus sp., Rhodococcus sp., Hydrogenovibrio sp., Rhodopseudomonas sp., Hydrogenobacter sp., Gordonia sp., Arthrobacter sp., Streptomycetes sp. Rhodobacter sp., and/or Xanthobacter.

In some embodiments, the microorganism is a cell of the class Actinobacteria. In some embodiments, the microorganism is strain of the suborder corynebacterineae (corynebacterium, gordoniaceae, mycobacteriaceae and nocardiaceae). In some embodiments, the microorganisms is a cell of the family of Nocardiaceae. In some embodiments, the microorganism or microorganisms are drawn from one or more of the following classifications: Corynebacterium, Gordonia, Rhodococcus, Mycobacterium and Tsukamurella. In some embodiments, the microorganism is a cell of the genus Rhodococcus. In some embodiments, the cell is a strain of a Rhodococcus sp., such as Rhodococcus opacus, Rhodococcus aurantiacus; Rhodococcus baikonurensis; Rhodococcus boritolerans; Rhodococcus equi; Rhodococcus coprophilus; Rhodococcus corynebacterioides; Nocardia corynebacterioides (synonym: Nocardia corynebacterioides); Rhodococcus erythropolis; Rhodococcus fascians; Rhodococcus globerulus; Rhodococcus gordoniae; Rhodococcus jostii; Rhodococcus koreensis; Rhodococcus kroppenstedtii; Rhodococcus maanshanensis; Rhodococcus marinonascens; Rhodococcus opacus; Rhodococcus percolatus; Rhodococcus phenolicus; Rhodococcus polyvorum; Rhodococcus pyridinivorans; Rhodococcus rhodochrous; Rhodococcus rhodnii; (synonym: Nocardia rhodnii); Rhodococcus ruber (synonym: Streptothrix rubra); Rhodococcus sp. RHA1; Rhodococcus triatomae; Rhodococcus tukisamuensis; Rhodococcus wratislaviensis (synonym: Tsukamurella wratislaviensis); Rhodococcus yunnanensis; or Rhodococcus zopfii. In some embodiments, the microorganism is strain Rhodococcus opacus DSM 43205 or DSM 43206. In some embodiments, the microorganism is strain Rhodococcus sp. DSM 3346.

In some embodiments, the composition comprises a microorganism (e.g., a microorganism of any of the microorganism genera or species described herein) that can naturally grow on H_{2/}CO₂ and/or syngas, and wherein the microorganism can naturally accumulate lipid to at least about 10%, 20%, 30%, 40%, 50% or more of the cell biomass by weight. In some embodiments, the microorganism (e.g., a microorganism of any of the microorganism genera or species described herein) has a native ability to send a high flux of carbon down the fatty acid biosynthesis pathway. In some embodiments, the microorganism exhibiting these traits is Rhodococcus opacus (e.g., DSM 43205 or DSM 43206 or DSM 44193) or Cupriavidus necator (e.g., DSM 531 or DSM 541).

In some embodiments, the microorganism is an oxyhydrogen or knallgas strain. In some embodiments, the microorganisms, or a composition comprising microorganisms, comprises one or more of the following knallgas microorganisms: Aquifex pyrophilus, Aquifex aeolicus, or other Aquifex sp.; Cupriavidus necator or Cupriavidus metallidurans or other Cupriavidus sp.; Corynebacterium autotrophicum or other Corynebacterium sp.; Gordonia desulfuricans, Gordonia polyisoprenivorans, Gordonia rubripertincta, Gordonia hydrophobica, Gordonia westfalica, or other Gordonia sp.; Nocardia autotrophica, Nocardia opaca, or other Nocardia sp.; purple non-sulfur photosynthetic bacteria, including but not limited to, Rhodobacter sphaeroides, Rhodopseudomonas palustris, Rhodopseudomonas capsulata, Rhodopseudomonas viridis, Rhodopseudomonas sulfoviridis, Rhodopseudomonas blastica, Rhodopseudomonas spheroides, Rhodopseudomonas acidophila, or other Rhodopseudomonas sp.; Rhodobacter sp., Rhodospirillum rubrum, or other Rhodospirillum sp.; Rhodococcus opacus or other Rhodococcus sp.; Rhizobium japonicum or other Rhizobium sp.; Thiocapsa roseopersicina or other Thiocapsa sp.; Pseudomonas facilis, Pseudomonas flava, Pseudomonas putida, Pseudomonas hydrogenovora, Pseudomonas hydrogenothermophila, Pseudomonas palleronii, Pseudomonas pseudoflava, Pseudomonas saccharophila, Pseudomonas thermophile, or other Pseudomonas sp.; Hydrogenomonas pantotropha, Hydrogenomonas eutropha, Hydrogenomonas facilis, or other Hydrogenomonas sp.; Hydrogenobacter thermophiles, Hydrogenobacter halophilus, Hydrogenobacter hydrogenophilus, or other Hydrogenobacter sp.; Hydrogenophilus islandicus or other Hydrogenophilus sp.; Hydrogenovibrio marinus or other Hydrogenovibrio sp.; Hydrogenothermus marinus or other Hydrogenothermus sp.; Helicobacter pylori or other Helicobacter sp.; Xanthobacter autotrophicus, Xanthobacter flavus, or other Xanthobacter sp.; Hydrogenophaga flava, Hydrogenophaga palleronii, Hydrogenophaga pseudollava, or other Hydrogenophaga sp.; Bradyrhizobium japonicum or other Bradyrhizobium sp.; Ralstonia eutropha or other Ralstonia sp.; Alcaligenes eutrophus , Alcaligenes facilis, Alcaligenes hydrogenophilus, Alcaligenes latus, Alcaligenes paradoxus, Alcaligenes ruhlandii, or other Alcaligenes sp.; Amycolata sp.; Aquaspirillum autotrophicum or other Aquaspirillum sp.; Arthrobacter strain 11/X, Arthrobacter methylotrophus, or other Arthrobacter sp.; Azospirillum lipoferum or other Azospirillum sp.; Variovorax paradoxus or other Variovorax sp.; Acidovorax facilis, or other Acidovorax sp.; Bacillus schlegelii, Bacillus tusciae, other Bacillus sp.; Calderobacterium hydrogenophilum or other Calderobacterium sp.; Derxia gummosa or other Derxia sp.; Flavobacterium autothermophilum or other Flavobacterium sp.; Microcyclus aquaticus or other Microcyclus sp.; Mycobacterium gordoniae or other Mycobacterium sp.; Paracoccus denitrificans or other Paracoccus sp.; Persephonella marina, Persephonella guaymasensis, or other Persephonella sp.; Renobacter vacuolatum or other Renobacter sp.; Seliberia carboxydohydrogena or other Seliberia sp., Streptomycetes coelicoflavus, Streptomycetes griseus, Streptomycetes xanthochromogenes, Streptomycetes thermocarboxydus, and other Streptomycetes sp.; Thermocrinis ruber or other Thermocrinis sp.; Wautersia sp.; cyanobacteria including but not limited to Anabaena oscillarioides, Anabaena spiroides, Anabaena cylindrica, or other Anabaena sp., and Arthrospira platensis, Arthrospira maxima, or other Arthrospira sp.; green algae including but not limited to Scenedesmus obliquus or other Scenedesmus sp., Chlamydomonas reinhardii or other Chlamydomonas sp., Ankistrodesmus sp., and Rhaphidium polymorphium or other Rhaphidium sp; as well as a consortium of microorganisms and/or organisms that includes oxyhydrogen microorganisms.

In some embodiments, the microorganisms, or a composition comprising microorganisms, comprises one or more of the following genera: Cupriavidus; Xanthobacter; Dietzia; Gordonia; Mycobacterium; Nocardia; Pseudonocardia; Arthrobacter; Alcanivorax; Rhodococcus; Streptomyces; Rhodopseudomonas; Rhodobacter; and Acinetobacter; as well as a consortium of microorganisms and/or organisms that includes one or more of these microorganisms.

In some embodiments, the microorganisms, or a composition comprising microorganisms, comprises one or more of the following: Arthrobacter methylotrophus DSM 14008; Rhodococcus opacus DSM 44304; Rhodococcus opacus DSM 44311; Xanthobacter autotrophicus DSM 431; Rhodococcus opacus DSM 44236; Rhodococcus ruber DSM 43338; Rhodococcus opacus DSM 44315; Cupriavidus metallidurans DSM 2839; Cupriavidus necator DSM 531; Cupriavidus necator DSM 541; Rhodococcus aetherivorans DSM 44752; Gordonia desulfuricans DSM 44462; Gordonia polyisoprenivorans DSM 44266; Gordonia polyisoprenivorans DSM 44439; Gordonia rubripertincta DSM 46039; Rhodococcus percolatus DSM 44240; Rhodococcus opacus DSM 43206; Gordonia hydrophobica DSM 44015; Rhodococcus zopfii DSM 44189; Gordonia westfalica DSM 44215, Xanthobacter autotrophicus DSM 1618; Xanthobacter autotrophicus DSM 2267; Xanthobacter autotrophicus DSM 3874; Streptomycetes coelicoflavus DSM 41471; Streptomycetes griseus DSM 40236; Streptomycetes sp. DSM 40434; Streptomycetes xanthochromogenes DSM 40111; Streptomycetes thermocarboxydus DSM 44293; Rhodobacter sphaeroides DSM 158. In some embodiments, the microorganisms, or a composition comprising microorganisms, comprises a consortium of microorganisms and/or organisms that includes one or more of these microorganisms or any of the microorganism genera or species disclosed herein.

A number of different microorganisms have been characterized that are capable of growing on carbon monoxide as an electron donor and/or carbon source (i.e., carboxydotrophic microorganisms). In some cases, carboxydotrophic microorganisms can also use H₂ as an electron donor and/or grow mixotrophically. In some cases, the carboxydotrophic microorganisms are facultative chemolithoautotrophs [Biology of the Prokaryotes, edited by J Lengeler, G. Drews, H. Schlegel, John Wiley & Sons, Jul. 10, 2009, is incorporated herein by reference in its entirety]. In some embodiments, the microorganisms or compositions comprising the microorganisms comprise one or more of the following carboxydotrophic microorganisms: Acinetobacter sp.; Alcaligenes carboxydus or other Alcaligenes sp.; Arthrobacter sp.; Azomonas sp.; Azotobacter sp.; Bacillus schlegelii or other Bacillus sp.; Hydrogenophaga pseudollava or other Hydrogenophaga sp.; Pseudomonas carboxydohydrogena, Pseudomonas carboxydovorans, Pseudomonas compransoris, Pseudomonas gazotropha, Pseudomonas thermocarboxydovorans, or other Pseudomonas sp.; Rhizobium japonicum or other Rhizobium sp.; and Streptomyces G26, Streptomyces thermoautotrophicus, or other Streptomyces sp.; In some embodiments, the microorganisms, or a composition comprising microorganisms, comprises a consortium of microorganisms and/or organisms that includes carboxydotrophic microorganisms, such as one or more of the above carboxydotrophic microorganisms.

In certain embodiments, a carboxydotrophic microorganism is used. In certain embodiments, a carboxydotrophic microorganism that is capable of chemolithoautotrophy is used. In certain embodiments, a carboxydotrophic microorganism that is able to utilize H₂ as an electron donor in respiration and/or biosynthesis is used.

In some embodiments, the microorganisms or compositions comprising the microorganisms comprise obligate and/or facultative chemoautotrophic microorganisms including one or more of the following: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Aquaspirillum sp.; Arcobacter sp.; Aureobacterium sp.; Bacillus sp.; Beggiatoa sp.; Butyribacterium sp.; Carboxydothermus sp.; Clostridium sp.; Comamonas sp.; Cupriavidus sp.; Dehalobacter sp.; Dehalococcoide sp.; Dehalospirillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobacterium sp.; Methanobrevibacter sp.; Methanococcus sp.; Methanococcoides sp.; Methanogenium sp.; Methanolobus sp.; Methanomicrobium sp.; Methanoplanus sp.; Methanosarcina sp.; Methanospirillum sp.; Methanothermus sp.; Methanothrix sp.; Micrococcus sp.; Nitrobacter sp.; Nitrobacteraceae sp., Nitrococcus sp., Nitrosococcus sp.; Nitrospina sp., Nitrospira sp., Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Siderococcus sp.; Streptomyces sp.; Sulfobacillus sp.; Sulfolobus sp.; Thermothrix sp., Thiobacillus sp.; Thiomicrospira sp.; Thioploca sp.; Thiosphaera sp.; Thiothrix sp.; Thiovulum sp.; sulfur-oxidizers; hydrogen-oxidizers; iron-oxidizers; acetogens; and methanogens; consortiums of microorganisms that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater. coal seams, deep sub-surface; waste water and sewage treatment plants; geothermal power plants, sulfatara fields, and soils; and extremophiles selected from one or more of thermophiles, hyperthermophiles, acidophiles, halophiles, and psychrophiles. In some embodiments, the microorganisms, or a composition comprising microorganisms, comprises a consortium of microorganisms and/or organisms that includes chemoautotrophic microorganisms, such as one or more of the above chemoautotrophic microorganisms.

In some embodiments, microorganisms are provided that are extremophiles that can withstand extremes in various environmental parameters, such as temperature, radiation, pressure, gravity, vacuum, desiccation, salinity, pH, oxygen tension, and chemicals. They include hyperthermophiles, such as Pyrolobus fumarii; thermophiles, such as Synechococcus lividis; mesophiles and psychrophiles, such as Psychrobacter, and/or extremely thermophilic sulfur-metabolizers such as Thermoproteus sp., Pyrodictium sp., Sulfolobus sp., and Acidianus sp.; radiation tolerant organisms such as Deinococcus radiodurans; pressure tolerant organisms including piezophiles or barophiles; desiccant tolerant and anhydrobiotic organisms including xerophiles, such as Artemia salina; microbes and fungi; salt tolerant organisms including halophiles, such as Halobacteriacea and Dunaliella salina; pH tolerant organisms including alkaliphiles, such as Natronobacterium, Bacillus firmus OF4, Spirulina spp., and acidophiles such as Cyanidium caldarium and Ferroplasma sp; gas tolerant organisms, which tolerate pure CO₂ including Cyanidium caldarium; and metal tolerant organisms including metalotolerants such as Ferroplasma acidarmanus and Ralstonia sp.

In certain embodiments, microorganisms provided herein comprise a cell line selected from eukaryotic plants, algae, cyanobacteria, green-sulfur bacteria, green non-sulfur bacteria, purple sulfur bacteria, purple non-sulfur bacteria, extremophiles, yeast, fungi, proteobacteria, engineered organisms thereof, and synthetic organisms. In certain embodiments, Spirulina is utilized.

In certain embodiments, green non-sulfur bacteria are utilized which include but are not limited to the following genera: Chloroflexus, Chloronema, Oscillochloris, Heliothrix, Herpetosiphon, Roseiflexus, and Thermomicrobium.

In certain embodiments, green sulfur bacteria are used which include but are not limited to the following genera: Chlorobium, Clathrochloris, and Prosthecochloris.

In certain embodiments, purple sulfur bacteria are used, which include but are not limited to the following genera: Allochromatium, Chromatium, Halochromatium, Isochromatium, Marichromatium, Rhodovulum, Thermochromatium, Thiocapsa, Thiorhodococcus, and Thiocystis.

In certain embodiments, purple non-sulfur bacteria are used which include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

In some embodiments, the microorganism is a methanotroph and/or a methylotroph. In some embodiments, the microorganism is in the genus Methylococcus. In some embodiments, the microorganism is Methylococcus capsulatus. In some embodiments, the microorganism is a methylotroph. In some embodiments, the microorganism is in the genus Methylobacterium. In some embodiments, the microorganism is drawn from one or more of the following species: Methylobacterium zatmanii; Methylobacterium extorquens; Methylobacterium chloromethanicum. In some embodiments, compositions are provided wherein the microorganism is a hydrogen-oxidizing chemoautotroph and/or a carboxydotroph and/or a methylotroph and/or methanotroph.

In certain embodiments, the microorganisms grow heterotrophically, utilizing multi-carbon organic molecules as carbon sources, such as, but not limited to sugars such as but not limited to glucose and/or fructose. In some embodiments, the microorganism is capable of growing on untreated crude glycerol and/or glucose and/or sucrose and/or cane juice and/or high fructose corn syrup and/or corn starch and/or cellulosic biomass and/or methanol and/or acetate as the sole electron donor and carbon source. In some embodiments, the microorganism is able to grow mixotrophically on an organic carbon source and using an inorganic electron donor or carbon source.

In certain embodiments, microorganisms provided herein comprise one or more of eukaryotic plants, algae, cyanobacteria, green-sulfur bacteria, green non-sulfur bacteria, purple sulfur bacteria, purple non-sulfur bacteria, extremophiles, archaea, yeast, fungi, proteobacteria, engineered organisms thereof, and synthetic organisms.

In certain embodiments, the microorganisms are naturally occurring and/or non-genetically modified (non-GMO) microorganisms and/or non-pathogenic and/or rely on specific environmental conditions provided by the bioprocesses that are absent from the surrounding environment.

In certain embodiments, the microorganisms or consortium of microorganisms are isolated from environmental samples and enriched with desirable microorganisms using methods known in the art of microbiology, for example, growth in the presence of targeted electron donors, including, but not limited to, one or more of: hydrogen, CO, syngas and/or methane, and/or electron acceptors including, but not limited to, one or more of oxygen, nitrate, ferric iron, and/or CO₂, and/or environmental conditions (e.g., temperature, pH, pressure, dissolved oxygen (DO), salinity, the presence of various impurities and pollutants, etc.).

In certain embodiments, the microorganisms or consortium of microorganisms comprise bacteria, fungi (e.g., yeast), and/or other microbial cells used to process sugar feedstocks into useful organic compounds such as proteins and amino acids in heterotrophic fermentation systems.

In certain embodiments, the microorganisms or consortium of microorganisms comprise probiotic microorganisms. In certain embodiments, the microorganisms or consortium of microorganisms comprise “generally recognized as safe” (GRAS) microorganisms and/or organisms. In certain embodiments, the microorganisms or organisms or consortium of microorganisms comprise yeast including, but not limited to, one or more of the following: Candida humilis; Candida milleri; Debaryomyces hansenii; Kazachstania exigua (Saccharomyces exiguous); Saccharomyces cerevisiae; Saccharomyces florentinus; Torulaspora delbrueckii; Trichosporon beigelli; and/or comprise fungi including, but not limited to, one or more of the following—Aspergillus oryzae; Aspergillus sojae; Fusarium venenatum A3/5; Neurospora intermedia var. oncomensis; Rhizopus oligosporus; Rhizopus oryzae; Aspergillus luchuensis—and/or comprise bacteria including one or more of the following—Bacillus amyloliquefaciens; Bacillus subtilis; Bifidobacterium animalis (lactis); Bifidobacterium bifidum; Bifidobacterium breve; Bifidobacterium longum; Lactobacillus acidophilus; Lactobacillus brevis; Lactobacillus casei; Lactobacillus delbrueckii subsp. bulgaricus; Lactobacillus fermentum; Lactobacillus helveticus; Lactobacillus kefiranofaciens; Lactobacillus lactis; Lactobacillus plantarum; Lactobacillus rhamnosus; Lactobacillus reuteri; Lactobacillus sakei; Lactobacillus sanfranciscensis; Lactococcus lactis (Streptococcus lactis, Streptococcus lactis subsp. diacetylactis); Leuconostoc; Leuconostoc carnosum; Leuconostoc cremoris; Leuconostoc mesenteroides; Pediococcus; Propionibacterium freudenreichii; Arthrospira (Spirulina) platensis; Streptococcus faecalis; Streptococcus thermophilus.

The protein containing biomass from which the protein hydrolysate composition is derived may be produced by a consortium of different species of microorganisms and/or multi-cellular organisms. In some embodiments, the consortium comprises one or more of: an oxyhydrogen microorganism; a carboxydotroph; a methanotroph; a methylotroph; a chemoautotroph; a photoautotroph; and a heterotroph.

In some embodiments, the protein hydrolysate also includes one or more vitamin(s) produced by the organisms from which the hydrolysate was derived. In some non-limiting embodiments, the microorganism is Cupriavidus necator DSM 531 or DSM 541. In some non-limiting embodiments, the vitamin is a B vitamin, including but not limited to, B1, B2, and/or B12.

Any suitable methods may be used to culture the microorganisms. The microorganism may be grown under any suitable conditions, in an environment that is suitable for growth and production of biomass. In some embodiments, the microorganism may be grown in autotrophic culture conditions, heterotrophic culture conditions, or a combination of autotrophic and heterotrophic culture conditions. A heterotrophic culture may include a suitable source of carbon and energy, such as one or more sugar (e.g., glucose, fructose, sucrose, etc.). An autotrophic culture may include C1 chemicals such as carbon monoxide, carbon dioxide, methane, methanol, formate, and/ or formic acid, and/or mixtures containing C1 chemicals, including, but not limited to various syngas compositions or various producer gas compositions, e.g., generated from low value or waste sources of carbon and energy, such as, but not limited to, lignocellulosic energy crops, crop residues, bagasse, saw dust, forestry residue, food waste, municipal solid waste, sewage, waste carpet, biogas, landfill gas, stranded natural gas, or pet coke through the gasification, partial oxidation, pyrolysis, or steam reforming of said low value or waste carbon sources, that can be used by an oxyhydrogen microorganism or hydrogen-oxidizing microorganism or carbon monoxide oxidizing microorganism as a carbon source and an energy source. Suitable ways of culturing the microorganisms and generating a biomass for use in the present methods are described, e.g., in U.S. Pat. Nos. 9,157,058 and 9,556,462, and PCT international publication number WO2018/144965, each of which is hereby incorporated by reference. In some embodiments, the organism may be grown photosynthetically in a bioreactor, in a hydroponics system, in a greenhouse, or in a cultivated field, or may be collected from waste or natural sources.

In certain embodiments, the protein-containing biomass used in the production of protein hydrolysate is from microorganism(s) grown on one or more C1 substrate. In certain embodiments, the microorganism(s) are grown on CO₂, and/or dissolved forms of CO₂ in aqueous solution (e.g., CO₂ (aq), bicarbonate, carbonate), as the sole carbon source. In certain embodiments the microorganism(s) grown on CO₂ and/or dissolved CO₂, comprise microbial cells that are selected from one or more of the following genera: Cupriavidus, Rhodococcus, Hydrogenovibrio, Rhodopseudomonas, Hydrogenobacter, Gordonia, Arthrobacter, Streptomycetes, Rhodobacter, and/or Xanthobacter. In certain embodiments the protein containing biomass, from microorganism(s) grown on CO₂ and/or dissolved CO₂, comprises microbials cells that are selected from one or more autotrophic (e.g., chemoautotrophic, photoautotrophic), carboxydotrophic, methanotrophic, methylotrophic, and/or heterotrophic microorganisms described above. In certain embodiments, the protein containing biomass, from microorganism(s) grown on CO₂ and/or dissolved CO₂, comprises Cupriavidus necator cells. In certain such embodiments, the strain of Cupriavidus necator may include, but is not limited to, DSM 428, DSM 531, or DSM 541. In certain embodiments the protein containing biomass, from microorganism(s) grown on CO₂ and/or dissolved CO₂, comprises microbial cells of a PHB negative mutant or knock-out of a wild-type strain that naturally produces PHB. In certain said embodiments the mutant strain that is unable to produce PHB is derived from a wild-type strain that is able to produce PHB, using well known methods. In certain embodiments, the said PHB negative mutant or knock strain produces less PHB than the wild-type strain, and/or undetectable amounts of PHB. In certain such embodiments, the PHB negative mutant is Cupriavidus necator DSM 541.

In some embodiments the organism or consortium can be grown mixotrophically. In some such mixotrophic growth conditions, a growth substrate comprises H₂ gas along with one or more sugar(s) as a carbon source.

### IV. Compositions

Disclosed herein are protein hydrolysate compositions produced using methods of the present disclosure. The proteinaceous component of the composition may include free amino acids, oligopeptides, and/or polypeptides that are about 25 kD or smaller, e.g., about 20 kD or smaller, about 15 kD or smaller, about 10 kD or smaller, about 5 kD or smaller, about 3 kD or smaller, or about 2 kD or smaller. The protein content of the composition may be analyzed by measuring the total amino acid content, using any suitable method (e.g., liquid chromatography). In some embodiments, the protein-rich organic content includes an amount of amino acids, weight by weight of the organic content (w/w), of about 10% or more, e.g., about 20% or more, about 30% or more, about 40% or more, about 50% or more, about 60% or more, about 70% or more, about 80% or more, including about 90% or more. In some embodiments, the protein-rich organic content includes an amount of amino acids, weight by weight of the organic content (w/w), in a range of about 10% to about 98%, e.g., about 20% to about 98%, about 30% to about 98%, about 40% to about 98%, about 50% to about 95%, about 60% to about 95%, about 70% to about 95%, including about 75% to about 95%. The total organic content may be measured using any suitable method.

In some embodiments, the protein hydrolysate includes an amount of amino acids, weight by weight of the total dry weight (w/w), of about 10% or more, e.g., about 20% or more, about 30% or more, about 40% or more, about 50% or more, about 60% or more, about 70% or more, about 80% or more, about 85% or more, including about 90% or more. In some embodiments, the protein hydrolysate has an amino acids content, weight by weight of the total dry weight (w/w), that is greater than or equal to the w/w amino acid content of the starting biomass. In some embodiments, the protein hydrolysate has an organic content weight by weight of the dry biomass content (w/w), of about 10% or more, e.g., about 20% or more, about 30% or more, about 40% or more, about 50% or more, about 60% or more, about 70% or more, or about 80% or more, including about 90% or more.

In some embodiments, the protein hydrolysate has an ash content weight by weight of the dry biomass content (w/w) of about 40% or less, e.g., about 30% or less, about 20% or less, about 10% or less, about 7% or less, or about 5% or less. In some embodiments, the protein hydrolysate has an ash content weight by weight of the dry biomass content (w/w) of about 10%. In some embodiments the ash content of the protein hydrolysate is about 5%. In certain embodiments, the protein hydrolysate has less than 1% higher ash content than the starting biomass. In certain embodiments, the protein hydrolysate has a % ash content that is less than or equal to the starting biomass. The ash content of a protein hydrolysate or biomass may be determined by well known methods, such as placing a protein hydrolysate or biomass sample in a tared crucible, and running an ash cycle in a muffle furnace

In some embodiments, the protein hydrolysate composition is free or substantially free of chelating agents (e.g., EDTA, EGTA, etc.), chaotropic agents (e.g., urea, phenol, etc.) and/or surfactants (e.g., SDS, etc.). In some embodiments, the protein hydrolysate composition is free or substantially free of synthetic chelating agents, chaotropic agents, and/or surfactants. In some embodiments, where the protein hydrolysate composition is produced from a microbial source without using any chelating agents, chaotropic agents and/or surfactants, the protein hydrolysate composition contains no detectable amount of chelating agents, chaotropic agents and/or surfactants, e.g., no detectable amount of exogenous chelating agents, chaotropic agents and/or surfactants.

In some embodiments, the protein hydrolysate composition has a nitrogen content of about 5% (w/w) or more, e.g., about 6% (w/w) or more, about 7% (w/w) or more, about 8% (w/w) or more, about 9% (w/w) or more, about 10% (w/w) or more, including about 13% (w/w) or more, and in some embodiments, about 20% (w/w) or less, about 18% (w/w) or less, about 15% (w/w) or less, about 13% (w/w) or less, including about 11% (w/w) or less. In some embodiments, the protein hydrolysate composition has a nitrogen content in a range of about 5% (w/w) to about 20% (w/w), e.g., about 6% (w/w) to about 18% (w/w), about 7% (w/w) to about 15% (w/w), including about 8% (w/w) to about 15% (w/w). The nitrogen content may be measured using any suitable method.

In some embodiments, the protein hydrolysate composition has a phosphate content, expressed in P₂O₅ equivalent, of about 5% (w/w) or more, e.g., about 6% (w/w) or more, about 8% (w/w) or more, including about 10% (w/w) or more, and in some embodiments, about 15% (w/w) or less, e.g., about 13% (w/w) or less, about 11% (w/w) or less, including about 10% (w/w) or less. In some embodiments, the protein hydrolysate composition has a phosphate content, expressed in P₂O₅ equivalent, in a range of about 5% (w/w) to about 15% (w/w), e.g., about 6%) w/w) to 13% (w/w), including about 6% (w/w) to about 11% (w/w). The phosphate content may be measured using any suitable method.

In some embodiments, the protein hydrolysate composition has a potassium content, expressed in K₂O equivalent, of about 5% (w/w) or more, e.g., about 7% (w/w) or more, about 10% (w/w) or more, about 12% (w/w) or more, including about 15% (w/w) or more, and in some embodiment, about 20% (w/w) or less, e.g., about 19% (w/w) or less, about 18% (w/w) or less, about 16% (w/w) or less, including about 15% (w/w) or less. In some embodiments, the protein hydrolysate composition has a potassium content, expressed in K₂O equivalent, in a range of about 5% (w/w) to about 20% (w/w), e.g., about 7% (w/w) to about 19% (w/w), including about 10% (w/w) to about 19% (w/w). The potassium content may be measured using any suitable method.

In some embodiments, the protein hydrolysate composition has an NPK (nitrogen, phosphate, potassium) content by wt % of at least 5:5:5.

In some embodiments, the protein hydrolysate composition has a sodium content of about 1% (w/w) or less, e.g., about 0.8% (w/w) or less, about 0.6% (w/w) or less, about 0.4% (w/w) or less, including about 0.3% (w/w) or less. In some embodiments, the protein hydrolysate composition has a chloride content of about 1% (w/w) or less, e.g., about 0.8% (w/w) or less, about 0.6% (w/w) or less, about 0.4% (w/w) or less, including about 0.3% (w/w) or less. The protein hydrolysate composition may be substantially free of manganese, calcium, copper and/or zinc. In some embodiments, the protein hydrolysate composition does not include detectable quantities of manganese, calcium, copper and/or zinc. In some embodiments, the protein hydrolysate composition includes one or more of manganese, calcium, copper and/or zinc.

The protein hydrolysate composition may be in liquid form, a suspension or slurry, or may be substantially dry. In some embodiments, the protein hydrolysate composition is in liquid form. In some embodiments, the protein hydrolysate composition is a dry powder, e.g., lyophilized powder. In some embodiments, the protein hydrolysate composition is a slurry, suspension, or emulsion.

### V. Uses of Protein Hydrolysate Compositions

Also disclosed herein are methods of using the protein hydrolysate compositions described herein. The protein hydrolysate compositions of the present disclosure may find use in various agricultural or horticultural settings. In some embodiments, the protein hydrolysate composition is used as a biostimulant and/or plant nutrient, or precursor thereof. As a biostimulant, the protein hydrolysate composition may promote the growth and/or health of a plant or livestock, or another microorganism or organism or cell culture, when provided or administered. In some embodiments, the protein hydrolysate composition is provided to a plant by foliar or soil application. In some embodiments, the protein hydrolysate composition is provided to a plant by fertigation. In some embodiments, the protein hydrolysate composition is provided within a hydroponic, aeroponic, or aquaponic system. In some embodiments, the protein hydrolysate produced according to the present invention may be used in place of an animal derived protein hydrolysate. In some embodiments, the protein hydrolysate provides a vegan substitute for a fish hydrolysate or fish emulsion.

The protein hydrolysate composition may serve as an ingredient for or precursor of a biostimulant product and/or plant nutrient, and may be combined with any other suitable component to form a plant supplement that supports plant health and/or growth. In some embodiments, the protein hydrolysate composition is combined with a preservative. Any suitable preservative may be used. Suitable preservatives include, without limitation, citric acid, benzoic acid, propylene glycol, propionic acid, sorbic acid, zinc sulfate, iron sulfate, copper sulfate, and/or silver chloride. In some embodiments, the protein hydrolysate composition is combined with a fertilizer. In some embodiments, the protein hydrolysate composition is combined with trace minerals, including, without limitation, one or more of iron, copper, zinc, boron, manganese, calcium, molybdenum, and magnesium. In some embodiments, the protein hydrolysate composition is combined with an herbicide, pesticide, and/or fungicide. Suitable herbicides, pesticides, and/or fungicides include, without limitation, thiophanate methyl, chlorothalonil, captan, piperalin, fenarimol, metalaxyl, triforine, ethoxy thialdiazole, pyretin, algicide, oryzalin, aldxylarypolyethoxyethanol, glyphosate, and/or naphthalene.

In some embodiments, the protein hydrolysate composition is provided to promote growth of a food crop. In certain such embodiments, the food crop comprises at least one member selected from a fruit, a vegetable, a tuber, and a grain. Examples of agricultural products include, but are not limited to, vegetables such as broccoli, cauliflower, globe artichoke, peas, beans, kale, collard greens, spinach, arugula, beet greens, bok choy, chard, choi sum, turnip greens, endive, lettuce, mustard, greens, watercress, garlic chives, gai Ian, leeks, brussels sprouts, capers, kohlrabi, celery, rhubarb, cardoon, Chinese celery, lemon grass, asparagus, bamboo shoots, galangal, ginger, soybean, mung beans, urad, carrots parsnips, beets, radishes, rutabagas, turnips, burdocks, onions, shallots, leeks, garlic, green beans, lentils, and snow peas; fruits, such as tomatoes, cucumbers, squash, zucchinis, pumpkins, melons, peppers, eggplant, tomatillos, christophene, okra, breadfruit, avocado, blackcurrant, redcurrant, gooseberry, guava, lucuma, chili pepper, pomegranate, kiwifruit, grapes, cranberry, blueberry, orange, lemon, lime, grapefruit, blackberry, raspberry, boysenberry, pineapple, fig, mulberry, hedge apple, apple, rose hip, and strawberry; nuts such as almonds, pecans, walnuts, brazil nuts, candlenuts, cashew nuts, gevuina nuts, horse-chestnuts, macadamia nuts, malabar chestnuts, mongongo, peanuts, pine nuts, and pistachios; tubers such as potatoes, sweet potatoes, cassava, yams, and dahlias; and cereals or grains such as maize, rice, wheat, barley, sorghum, millet, oats, rye, triticale, fonio, buckwheat, and quinoa. In other embodiments, the crop is an ornamental crop. In certain such embodiments, the ornamental crop comprises at least one member selected from turfgrass, a tree, a shrub, and a flower. In other embodiments, the crop is a mushroom or fungus. Examples of fungal crops include but are not limited to: Agaricus bisporus (button, crimini, and portabella), Coprinus quadrifidus , Lepista nuda, and Pleurotus ostreatus (oyster mushrooms). In certain embodiments, a protein hydrolysate as described herein is applied to a mushroom or fungus, such as one or more of the following: Agaricus bisporus, Coprinus quadrifidus, Lepista nuda, and Pleurotus ostreatus.

In some embodiments, the protein hydrolysate composition is used as a nutrient source for another organism, e.g., animals, humans, cells (prokaryotic or eukaryotic cells), or as a precursor of such a nutrient source or an ingredient in forming such a nutrient source. In certain embodiments, hydrolysates produced as described herein are used as a supplement in cell cultures. Certain embodiments of this disclosure herein relate to hydrolysates with application in sport medicine and specifically, in certain non-limiting embodiments, consumption of said hydrolysate allows amino acids to be absorbed by the body more rapidly than intact proteins, thus maximizing nutrient delivery to muscle tissues. In certain embodiments, the hydrolysate is rich in antioxidants and/or L-aspartic acid and/or manganese and/or selenium. Certain embodiments relate to using the microbial hydrolysates, either directly or as an ingredient in pet food, including but not limited to food for mammals, such as dogs, cats, rabbits, small rodents, horses, etc., or poultry, such as chickens, turkeys, etc. In certain embodiments, the hydrolysate can also be used as a nutritional additive with a high added value for animal feed, more particularly, animal feed for livestock (e.g., cattle, sheep, goats, pigs, etc.), or aquaculture (e.g., fish, shellfish), or insects (e.g., bees) or invertebrates (e.g., worms), or heterotrophic microorganisms (e.g., yeast; E. coli), or for domestic animals or pets, or for human consumption.

Suitable uses for a protein hydrolysate composition of the present disclosure are described in, e.g., PCT international publication number WO2018/144965, which is incorporated by reference in its entirety.

Other features of the present disclosure will become apparent in the course of the following descriptions of examples. The following examples are intended to illustrate, but not limit, the invention.

### EXAMPLES

### Example 1: Protein Hydrolysate Produced from a Cupriavidus necator Culture

### Producing a Protein Hydrolysate from a Microbial Culture

A Cupriavidus necator strain was cultivated in a growth medium with glucose as carbon source. After growth, whole cell biomass was isolated from the growth medium and dried. A portion of the dried biomass was processed as follows:
1. 2 g of the dried biomass was suspended in 100 ml of water.
2. The suspended biomass was homogenized with IKA T25 Turrax stick at 15000 rpm for 1 min.
3. 10N KOH stock was added to raise the pH.
4. The alkaline biomass solution was autoclaved at 121° C. for 30 mins at slow exhaust.
5. After the autoclave cycle, autoclaved solution was cooled to room temperature.
6. The solution was neutralized with 1 M KH₂PO₄ buffer, pH 5.8, and the pH was brought down to ˜8.8.
7. 100 μl 1 mg/ml bacterial alkaline protease (made up in 10 mM tris buffer pH 7.4) was added to the neutralized solution.
8. The resulting reaction was digested overnight at 55° C. in a shaking water bath.
9. After enzymatic hydrolysis, the reaction was centrifuged, and the supernatant was separated from the pellet.

### Content Analysis of Protein Hydrolysate

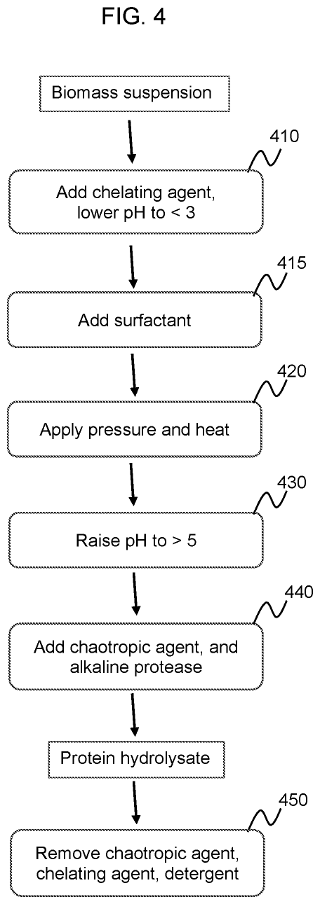

The supernatant and pellet fractions were analyzed on an SDS-PAGE gel (FIG. 6). The supernatant fraction contained most of the protein and polypeptide fragments, which were less than 15 kD.

FIG. 6: Gel image showing the pellet (Lane 1) and supernatant (Lane 2) fractions after the enzymatic hydrolysis. Lane 3 contains markers.

The supernatant from Example 1 was freeze-dried to produce a powder, which was analyzed for the following:
1. Nitrogen content
2. Ash and moisture level
3. Phosphorus, Phosphate, Potassium and potash content
4. Amino acids (all 20 amino acids including cysteine, methionine and tryptophan) content
5. Process yields and complete mass balances by weight.

### Example 2: Protein Hydrolysate Produced from a Cupriavidus necator Culture

A protein hydrolysate was produced from a C. necator strain culture as in Example 1. KOH was added to the suspended biomass to obtain a pH of 11.3.

The supernatant was freeze-dried to produce a powder, which was analyzed for the following:
1. Nitrogen content (Table 1)
2. Ash and moisture level (Table 1)
3. Phosphorus, Phosphate, Potassium and potash content (Tables 1 and 2)
4. Amino acids (all 20 amino acids including cysteine, methionine and tryptophan) content (Table 2)
5. Process yields and complete mass balances by weight.

TABLE 1
Content Analysis (% w/w)

| | |
|---|---|
| Moisture content | 4.0% |
| Organic matter | 80% |
| Ash | 16% |
| Crude Protein | 78% |
| Protein from amino acids | 73.6% |
| Amino acids as a % of Organic matter | 92% |
| Total Nitrogen | 12.6% |
| C:N ratio | 3.2 |
| Sulfur | 0.51% |
| Phosphate (P2O5) | 6.46% |
| Potash (K2O) | 7.6% |

TABLE 2
Amino Acids and Micro-Nutrients (% w/w)

| | |
|---|---|
| AMINO ACIDS | MICRO NUTRIENTS |

| | | | |
|---|---|---|---|
| Cysteine | 0.22% | Potassium | 5.49% |
| Methionine | 1.96% | Phosphorus | 2.99% |
| Tryptophan | 0.89% | Sodium | 0.56% |
| Alanine | 7.36% | Zinc | <20 ppm |
| Arginine | 5.13% | Boron | 107 ppm |
| Aspartic Acid + | 7.83% | Magnesium | 0.27% |
| Asparagine | | | |
| Glutamic Acid + | 8.82% | Iron ppm | 275 ppm |
| Glutamine | | | |
| Glycine | 4.71% | Calcium | <0.01% |
| Histidine | 1.45% | | |
| Isoleucine | 2.98% | | |
| Leucine | 6.35% | | |
| Lysine | 5.62% | | |
| Phenylalanine | 3.1% | | |
| Proline | 2.55% | | |
| Serine | 2.88% | | |
| Threonine | 3.85% | | |
| Tyrosine | 2.55% | | |
| Valine | 5.35% | | |

This protocol resulted in a protein hydrolysate produced from bacterial cell biomass. The protein hydrolysate (PH) contained a complex mixture of amino acids, proteins, and inorganic nutrients including NPK. The PH had very high % amino acids (w.r.t organic matter). Organic matter in this PH was 80%, and 92% of this organic matter was amino acids.

### Example 3: Protein Hydrolysate Production from a Second C. necator Culture

A second portion of the dried biomass derived from a C. necator strain culture was processed as in Example 1. KOH was added to the suspended biomass to 50 mM in the final volume.

The supernatant obtained was freeze dried and analyzed as in Example 2. The results of the analysis are shown in Tables 3 and 4.

TABLE 3
Content Analysis (% w/w)

| | |
|---|---|
| Moisture content | 2.6% |
| Organic matter | 64.3% |
| Ash | 33.1% |
| Crude Protein | 57% |
| Protein from amino acids | 48.97% |
| Amino acids as a % of Organic matter | 76% |
| Total Nitrogen | 9.12% |
| C:N ratio | 3.75 |
| Sulfur | 0.38% |
| Phosphate (P2O5) | 11% |
| Potash (K2O) | 18.7% |

TABLE 4
Amino Acids and Micro-Nutrients (% w/w)

| | |
|---|---|
| AMINO ACIDS | MICRO NUTRIENTS |

| | | | |
|---|---|---|---|
| Cysteine | 0.14% | Potassium | 14.28% |
| Methionine | 1.58% | Phosphorus | 4.82% |
| Tryptophan | 0.86% | Sodium | 0.3% |
| Alanine | 5.01% | Zinc | ND^{†} |
| Arginine | 2.99% | Boron | 136 ppm |
| Aspartic Acid + | 5.08% | Magnesium | 0.11% |
| Asparagine | | | |
| Glutamic Acid + | 5.84% | Iron ppm | 226 ppm |
| Glutamine | | | |
| Glycine | 3.44% | Calcium | <0.01% |
| Histidine | 1.08% | | |
| Isoleucine | 2.04% | | |
| Leucine | 4.43% | | |
| Lysine | 3.33% | | |
| Phenylalanine | 2.39% | | |
| Proline | 2.15% | | |
| Serine | 1.4% | | |
| Threonine | 1.83% | | |
| Tyrosine | 1.77% | | |
| Valine | 3.61% | | |

^{†}Not determined

### Example 4: Protein Hydrolysate Production from a Larger-Scale C. necator Culture

A whole cell biomass weighing about 310 g was obtained from a culture of C. necator. The process described in Example 1 was scaled up to process the larger biomass. The dried biomass was suspended in water at a ratio of 20 mg/ml. KOH was added to the suspension to 50 mM. The pH of the suspension after addition of KOH was 12.3. After autoclaving the pH was between 10.6 and 10.9. The autoclaved suspension was neutralized with phosphate buffer (KH₂PO₄) to pH 8.9-9.0. The pH after overnight hydrolysis was 8.01.

### Example 5: Effect of pH (1)

The amino acid and organic matter content was compared between the protein hydrolysate derived from two different base treatments. The protein hydrolysate from Example 2 (pH titrated to 11.3 before autoclaving) had a higher organic matter and amino acid content compared to a protein hydrolysate made using KOH added to 50 mM (pH ˜12) before autoclaving. In Table 5, the amino acid and nitrogen content was each measured against the dry weight of the protein hydrolysate.

TABLE 5
Amino acid content of various protein hydrolysates

| | Sample | | | | |
|---|---|---|---|---|---|
| | pH 11.3 | pH 12 | pH 12 | pH 12 | pH 12 |
| Nitrogen (%) | 12.48 | 9.99 | 10.13 | 10.03 | 10.19 |
| Amino acids (%) | 73.6 | 56.1 | 55.88 | 55.4 | 55.34 |
| Jones Factor | 5.84 | 5.61 | 5.51 | 5.52 | 5.43 |

### Example 6: Effect of pH (2)

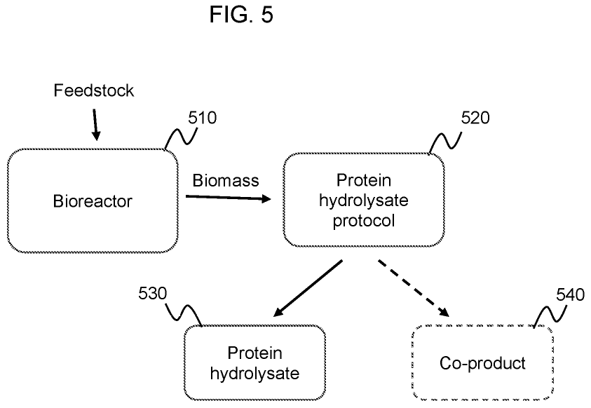

The pH of a biomass suspension obtained from a C. necator culture was adjusted to pH 9.6 with 10N NaOH. The alkaline suspension was autoclaved at 121° C. for 20 minutes with slow exhaust. The hydrolyzed suspension was centrifuged and the supernatant and pellet fractions were assayed for protein content by SDS-PAGE (FIG. 7). There was incomplete partitioning of protein into the supernatant.

FIG. 7: Gel image showing protein distribution between pellet and supernatant fractions in 5 independent samples. Sample 1: lanes 2-4; sample 2: lanes 5-7; sample 3: lanes 8-10; sample 4: lanes 11-13 (lanes for each sample correspond to input, pellet and supernatant fractions, respectively); sample 5: lanes 14 and 15 (lanes correspond to pellet and supernatant fractions, respectively). 5 g of wet biomass (lanes 2-13) or dry biomass (lanes 14-15) were treated. Samples 1, 3 and 5 were treated with base, samples 2 and 4 were not treated with base. Lane 1: Marker.

### Example 7: Effect of Neutralizing Buffer, Surfactant, Chelating Agent, and Chaotropic Agent

A set of samples, each sample having 0.3 g of dry biomass suspended and homogenized, was prepared. Samples were treated with 10N NaOH added to 0.1N, autoclaved for 10 minutes at 110° C. with fast exhaust to cool, neutralized with 1M potassium phosphate buffer at pH 5.8 (unless indicated otherwise), and digested with bacterial alkaline protease at 55° C. overnight.

All samples were centrifuged at 11,000 rpm for 15 minutes after the overnight enzyme treatment, and the supernatant was separated from the pellet. The different fractions were analyzed by SDS PAGE, Lowry Assay and OPA assay.

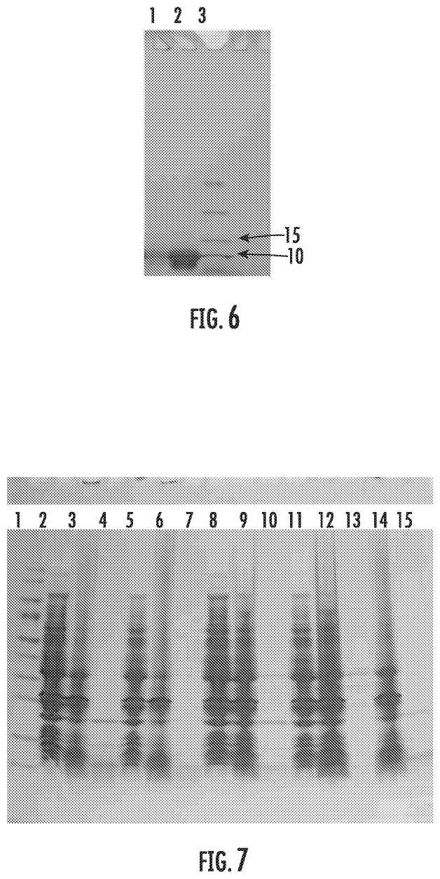

To test the effect of surfactant treatment, ammonium lauryl sulfate, Triton X-100, Tween 80, Tween 20, or Pluronic PF-68 were added to the biomass suspension to some samples before autoclaving (FIGS. 8A, 8B). Surfactants were added to a final amount of 10% relative to the dry weight of biomass. To test the effect of neutralizing buffer, some samples were neutralized with 1M Tris buffer pH 7.4 instead of potassium phosphate buffer (FIGS. 8A, 8B).

FIG. 8A: Gel image showing protein distribution between pellet and supernatant fractions for samples treated with Tris buffer, and different surfactants. Lane 1: marker; lane 2: SDS (pellet); lane 3: SDS (supernatant); lane 4: no surfactant (pellet); lane 5: no surfactant (supernatant); lane 6: ammonium lauryl sulfate (pellet); lane 7: ammonium lauryl sulfate (supernatant); lane 8: Triton X-100 (pellet); lane 9: Triton X-100 (supernatant); lane 10: Tween 80 (pellet); lane 11: Tween 80 (supernatant); lane 12: Tween 20 (pellet); lane 13: Tween 20 (supernatant); lane 14: Pluronic PF-68 (pellet); lane 15: Pluronic PF-68 (supernatant).

FIG. 8B: Gel image showing protein distribution between pellet and supernatant fractions for samples treated with potassium phosphate buffer, and different surfactants. Lane 1: marker; lane 2: SDS (pellet); lane 3: SDS (supernatant); lane 4: no surfactant (pellet); lane 5: no surfactant (supernatant); lane 6: ammonium lauryl sulfate (pellet); lane 7: ammonium lauryl sulfate (supernatant); lane 8: Triton X-100 (pellet); lane 9: Triton X-100 (supernatant); lane 10: Tween 80 (pellet); lane 11: Tween 80 (supernatant); lane 12: Tween 20 (pellet); lane 13: Tween 20 (supernatant); lane 14: Pluronic PF-68 (pellet); lane 15: Pluronic PF-68 (supernatant).

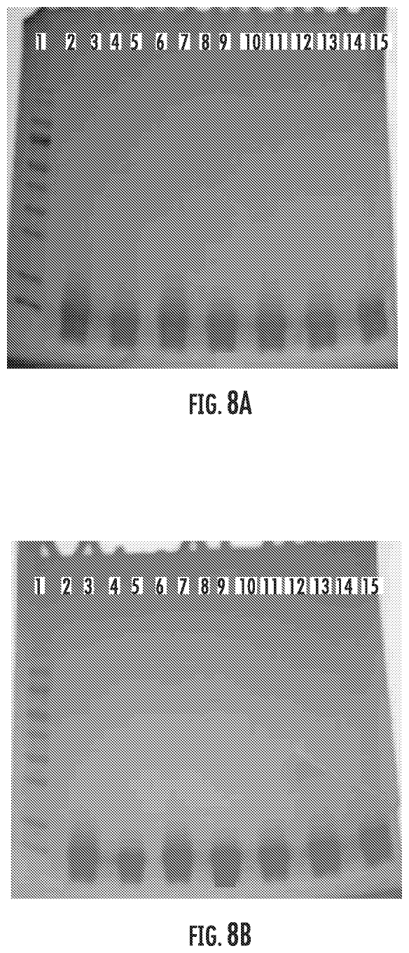

To test different bases, KOH was used instead of NaOH in some samples before autoclaving (FIG. 9). To test the effect of a chelating agent, EDTA was added to a final concentration of 5 mM in some samples before autoclaving (FIG. 9).

FIG. 9: Gel image showing protein distribution between pellet and supernatant fractions for samples treated with NaOH or KOH, and different combinations of EDTA and urea. NaOH-treated samples (left): Lane 1: marker; lane 2: EDTA, urea (pellet); lane 3: EDTA, urea (supernatant); lane 4: EDTA (pellet); lane 5: EDTA (supernatant); lane 6: urea (pellet); lane 7: urea (supernatant); lane 8: control (pellet); lane 9: control (supernatant); lane 10: marker (low range); KOH-treated samples (right): Lane 1: marker; lane 2: EDTA, urea (pellet); lane 3: EDTA, urea (supernatant); lane 4: EDTA (pellet); lane 5: EDTA (supernatant); lane 6: urea (pellet); lane 7: urea (supernatant); lane 8: control (pellet); lane 9: control (supernatant); lane 10: marker (low range).

Conclusion: KOH is suitable as a basic reagent. Urea and EDTA treatment increased total protein and supernatant protein yields. Chaotropic and chelating agents such as urea or EDTA may be removed by buffer exchange from the resultant supernatant fraction.

### Example 8: Biostimulant Effects of a Protein Hydrolysate

The biostimulant effect of the protein hydrolysate of the present disclosure is tested on various plants, such as turfgrass, radishes, lettuce, etc., as well as mushrooms. The growth of plants or mushrooms is compared with and without application of the protein hydrolysate. In some cases, the protein hydrolysate is combined with other active components to make a plant supplement, which is applied to the plant. The plant may be grown in soil, or hydroponically. The protein hydrolysate may be provided to the soil, or by foliar application. The biostimulant effect may be measured by growth density (turfgrass), weight, nitrogen uptake, or chlorophyll content, etc.

### Example 9: Protein Hydrolysate Produced from a Cupriavidus necator Culture

A Cupriavidus necator strain was cultured in a growth medium with fructose as the carbon source. After growth, whole cell biomass (WCB) was isolated from the growth medium and dried. WCB was processed as follows:
1. 32 g of the dried WCB was suspended in 350 ml of water.
2. The suspended biomass was homogenized with an Ultra Turrex Stick for 1 min at 13,200 RPM for 1 minute.
3. 13.51 ml of phosphoric acid (H₃PO₄) was added at a concentration of 85% (14.8M).
4. The suspension was transferred to pressure tubes.
5. The acidic biomass suspension was autoclaved at 121° C., 15 psi, for 1 hour.
6. After autoclaving, the suspension was cooled to room temperature. The pH of the suspension after autoclaving was 1.5.
7. The solution was neutralized with 2.2 g Ca(OH)₂ per 50 ml of autoclaved acidic suspension. Neutralization was performed overnight at 4° C. After neutralization, the pH was 5.5.
8. The neutralized suspension was centrifuged at 9603 RPM for 20 minutes at 4° C.
9. The supernatant was collected.
10. The supernatant and pellet were frozen at −80° C. for 3 hours and then transferred to a lyophilizer (Labconco Freezone 4.5 L, −50 C).
11. The samples were lyophilized until dry.

### Gel Analysis

The lyophilized acid hydrolysis sample (“test”) was analyzed on SDS-PAGE (Novex 4-20% Tris-Glycine), in comparison to a lyophilized sample that was not subjected to acid hydrolysis or calcium hydroxide neutralization (“control”). The acid hydrolysate exhibited a dense, brighter band than the pellet fraction, indicating that a significant amount of protein had been extracted and solubilized into the supernatant.

### Content Analysis of Protein Hydrolysate

The lyophilized acid hydrolysis sample (“test”) was analyzed for the following, in comparison to a lyophilized sample that was not subjected to acid hydrolysis or calcium hydroxide neutralization (“control”):
1. Moisture, ash, and nitrogen (Table 6)
2. Mass balance
3. Nitrogen balance
4. % Nitrogen solubility

Percent moisture, ash, and nitrogen on lyophilized dry mass are shown in Table 6.

TABLE 6

| Sample | Moisture % | Ash % | Nitrogen % |
|---|---|---|---|
| Test supernatant | 10.26 | 25.00 | 11.303 |
| Control supernatant | 10.02 | 15.79 | 11.029 |
| Test pellet | 8.37 | 4.99 | 11.471 |
| Control pellet | 3.66 | 4.10 | 13.796 |

### Example 10: Production of Low Ash Hydrolysates Using Defatted Whole Cell Biomass

Several methods were attempted to raise the amino acid content of protein hydrolysates and decrease the inorganics or ash content. These methods were tested on defatted biomass.

Defatting of the whole cell biomass was performed by suspending biomass in ethanol at a ratio of 3.75 mL per 1 g of biomass after which 0.5 mL NH₄OH was added per gram of biomass. This solution was stirred on a magnetic stir plate for 30 minutes before being filtered through Whatman 4 filter paper. The lipid containing filtrate (EtOH/NH₄OH) was then dried on a sand bath at 50° C. The remaining defatted biomass was air dried overnight in a fume hood before being oven dried at 40° C. for 4-6 hours. This dried, defatted biomass was then used for subsequent hydrolysis reactions. The following variations were attempted on the defatted biomass.

Enzyme only digestion: A 2% solids solution was made with defatted biomass and distilled water. This solution was homogenized and adjusted to a pH of 9 with 10N KOH and bacterial alkaline protease was added and incubated overnight in a shaking water bath at 55° C. The hydrolysate was then centrifuged for 30 minutes at 26,000×g and the supernatant and pellet fractions were separated and freeze dried.

Using solid Ca(OH)₂ to precipitate the inorganics: A 2% solids solution was made with defatted biomass and distilled water. This solution was homogenized and adjusted to a pH of 11 or 12 with Ca(OH)₂ and autoclaved for 10 minutes at 110° C. before being adjusted to a pH of 9 with H₃PO₄. Bacterial alkaline protease enzyme was then added and incubated overnight in a shaking water bath at 55° C. The hydrolysate was then centrifuged for 30 minutes at 26,000×g and the supernatant and pellet fractions were separated and freeze dried.

In Table 7, Mass, nitrogen yields and ash content of each condition are presented. The enzyme digested samples have the lowest ash content, while the ash content of the pellet for the chemically hydrolyzed samples is high (˜22-32%) as it contains the precipitated inorganics. The protein hydrolysates derived from chemical hydrolysis supernatants have low ash comparable to the starting material, as observed in Table 7, and high amino acid content, as observed in table 8.

TABLE 7
Mass and Nitrogen yields for hydrolysis of defatted biomass

| Condition | Sample | Mass yield | N yields | % ash |
|---|---|---|---|---|
| No digestion | Defatted whole | N/A | N/A | 7.08% |
| | cell biomass | | | |
| Enzyme only | pellet | 21.33% | 18.79% | 7.78% |
| digestion | supernatant | 70.27% | 78.14% | 12.01% |
| pH 11, Ca(OH)₂ + | pellet | 30.46% | 22.55% | 22.65% |
| H₃PO₄ digestion | supernatant | 56.40% | 60.14% | 10.49% |
| pH 12, Ca(OH)₂ + | pellet | 32.97% | 22.55% | 31.20% |
| H₃PO₄ digestion | supernatant | 74.20% | 76.87% | 11.60% |

TABLE 8
Amino acid and mineral analysis for pH 12 digestion

| | Starting whole | pH 12 |
|---|---|---|
| Type of sample | cell biomass | supernatant |
| % N | 11.87 | 13.57 |
| Crude Protein (6.25 × % N) | 74.17 | 84.81 |
| Total amino acid analysis | 54.02 | 69.92 |
| (% of total dry weight (w/w)) | | |
| | % of total dry | % of total dry |
| Amino Acid | weight (w/w) | weight (w/w) |
| Cysteine | 0.37 | 0.25 |
| Methionine (I) | 1.39 | 1.57 |
| Tryptophan (I) | 0.52 | 0.65 |
| Alanine | 5.18 | 5.99 |
| Arginine | 4.01 | 4.96 |
| Aspartic Acid | 5.42 | 7.35 |
| Glutamic Acid | 6.7 | 8.88 |
| Glycine | 3.29 | 4.01 |
| Histidine (I) | 1.13 | 1.41 |
| Isoleucine (I) | 2.24 | 2.8 |
| Leucine | 4.78 | 5.86 |
| Lysine (I) | 4.13 | 5.33 |
| Phenylalanine (I) | 2.39 | 3.23 |
| Proline | 2.19 | 4.52 |
| Serine | 2.08 | 2.73 |
| Threonine (I) | 2.84 | 3.55 |
| Tyrosine | 1.66 | 2.28 |
| Valine (I) | 3.7 | 4.55 |
| Ca % | 0.19 | 1.43 |
| Cu ppm | ND | 3.36 |
| Fe ppm | 1655.44 | 28.62 |
| POTASSIUM % | 0.85 | 1.29 |
| Mg % | 0.14 | 0.13 |
| Mn ppm | 1.44 | N.D. |
| Na % | 0.83 | 1.18 |
| PHOSPHORUS % | 2.11 | 1.17 |
| S % | 0.51 | 1.34 |
| Zn PPM | 7.1 | N.D. |

### Example 11: Low Ash Protein Hydrolysate Produced from a Cupriavidus necator Culture

A Cupriavidus necator PHB negative mutant strain (DSM 541) was cultivated in a growth medium with CO₂ as a carbon source. After growth, whole cell biomass was isolated from the growth medium dried by lyophilization. A portion of the dried biomass was processed as follows:

Defatting the whole cell biomass: WCB was defatted (lipids extracted out) with ammonium hydroxide and methanol (1:1:0.4, WCB:NH₄OH:MeOH) by stirring the mixture for an hour in a fume hood in a tightly capped container, then vacuum filtered with Whatman 4 filter paper. Filtrate collected had lipids extracted out. The retentate on the filter was defatted and dried at 40° C. in an incubator overnight.

Protein hydrolysis on defatted mass with H₃PO₄ and Ca(OH)₂: Solid loading of 8% of defatted dried mass was used and rehydrated with the required amount of DI water, then mixed well with a Turret Stick at 15000 rpm for 1 min. Strong phosphoric acid (14.8 M) was added to make the final reaction concentration of 0.5 M. The reaction was performed in a pressure tube with a tightly fitted screw cap, then autoclaved at 121 C, 15 psi for 1 hr and cooled down the reaction in a fume hood. Calcium hydroxide was added to neutralize to pH 8-9, and the pH checked with a pH meter. Protein hydrolysate (supernatant) was separated by centrifugation at 10000×g for 20 min at 7° C. The obtained liquid protein hydrolysate was freeze dried to produce dry powder. Measured ash content of the hydrolysate was 7.04%. Ash content was measured by placing a minimum of 300 mg protein hydrolysate powder in a tared crucible, and running an ash cycle in a muffle furnace, and also by external lab analysis (SGS, North America).

### Example 12: Low Ash Protein Hydrolysate Produced from a Cupriavidus necator Culture

A Cupriavidus necator strain (-PHB) was cultivated in a growth medium with CO₂ as a carbon source. After growth, whole cell biomass was isolated from the growth medium and dried by lyophilization. A portion of the dried biomass was processed as follows:

Defatting the whole cell biomass: WCB was defatted (lipids extracted out) with ammonium hydroxide and methanol (1:1:0.4, WCB:NH₄OH:MeOH) by stirring the mixture for an hour in a fume hood in a tightly capped container, then vacuum filtered with Whatman 4 filter paper. Filtrate collected had lipids extracted out. The retentate on the filter was defatted and dried at 40° C. in an incubator overnight.

Protein hydrolysis with NH₄OH and CO₂: Solid loading of 2% of defatted dried mass was used and rehydrated with the required amount of DI water, then mixed well with a Turret Stick at 15,000 rpm for 1 min. The pH of the reaction mix was increased to 10.85 by addition of NH₄OH 28%-30% (pre-made) solution in a fume hood. The mixture was transferred to the pressure tube (size: 120 mL) with 50 mL working volume, and autoclaved at 110° C., 10 min, slow exhaust. The pH of the solution post autoclave was 10.82. The pH was decreased to 9 with CO₂ bubbling by inserting a cannula/18G needle for 10-20 min. Enzyme digestion was performed with Bacterial Alkaline Protease at 55° C., 110 rpm overnight. The supernatant, containing soluble hydrolyzed proteins, and pellet (PHB rich crude pellet) were separated by centrifugation at 20000×g, 20 min, 5° C. The protein hydrolysate was freeze dried.

Measured ash content of the protein hydrolysate was 5%. Ash content was measured by placing a minimum of 300 mg of protein hydrolysate powder in a tared crucible, and running an ash cycle in a muffle furnace, and also by external lab analysis (SGS, North America).

Results are shown in Table 9.

TABLE 9

| % Total Amino Acids | 84.96 |
|---|---|
| Sample form | Dry powder |
| % Ash | 4.99 |
| Amino Acid | % of total dry weight (w/w) |
| Cysteine | 0.13 |
| Methionine | 1.62 |
| Tryptophan | 0.95 |
| Alanine | 7.7 |
| Arginine | 6.19 |
| Aspartic Acid | 6.1 |
| Glutamic Acid | 10.34 |
| Glycine | 5.11 |
| Histidine | 1.59 |
| Isoleucine | 3.04 |
| Leucine | 7.36 |
| Lysine | 6.28 |
| Phenylalanine | 4.95 |
| Proline | 3.77 |
| Serine | 6.1 |
| Threonine | 3.8 |
| Tyrosine | 4.41 |
| Valine | 5.52 |

### Example 13: Low Ash Protein Hydrolysate Produced from a Cupriavidus necator Culture

A Cupriavidus necator PHB negative mutant strain (DSM 541) was cultivated in a growth medium either with CO₂ or sugar as a carbon source. After growth, the whole cell biomass was isolated from the growth medium and dried by lyophilization. A portion of the dried biomass was processed as follows:

Defatting the whole cell biomass: Dry whole cell biomass was defatted via treatment with ammonium hydroxide and ethanol (1:4:0.5 w/v). The biomass and solvent slurry was stirred for 30 minutes in a tightly capped glass bottle before being vacuum filtered through Whatman 4 filter paper in a fume hood. The filtrate collected contained the lipid fraction while the defatted retentate was air dried overnight before being dried at 40° C. in an incubator for 4-6 hours.

Protein hydrolysis on defatted biomass with Ca(OH)₂ and H₃PO₄: Dried defatted biomass was resuspended in DI water to a final concentration of 2%. The biomass solution was mixed with IKA Ultra-Turax at 15000 rpm until fully and homogeneously resuspended. The biomass solution was brought to a pH of 11 by addition of Ca(OH)₂. The solution was transferred to a glass media bottle and autoclaved at 110° C. for ten minutes and subsequently cooled to room temperature. The solution was neutralized to pH 9 using H₃PO₄. Bacterial alkaline protease (Sigma P8038) was added to the solution at a concentration of 2.6 active units/g biomass. The biomass solution was placed in a 55° C. shaking water bath overnight. After 16-24 hour digestion, the enzyme was inactivated by incubating the slurry in a 95° C. water bath for ten minutes. The biomass slurry was then cooled to room temperature and the protein hydrolysate (supernatant fraction) was separated by centrifugation at 26000×g for 30 minutes at 7° C. The resulting protein hydrolysate solution was frozen in a −80° C. freezer before being lyophilized. The moisture, ash, and N content of the dried powder was determined and the protein profile was analyzed via SDS-PAGE analysis. There were no proteins above 2000 Dalton present in the protein hydrolysate, and the resulting ash content was 10.5%.

### Example 14: Demonstration of Biostimulant Effect of Protein Hydrolysate Produced Using Acid Hydrolysis on Brassica rapa Seeds

Protein hydrolysate produced using phosphoric acid incubation, followed by a neutralization with Calcium hydroxide to precipitate the phosphate ion and neutralize the solution to pH 5, was tested for its impact as a biostimulant using Brassica rapa (turnip) seeds according to OECD GUIDELINES FOR THE TESTING OF CHEMICALS 208 and ISO 11269-1, 2.

10 Seeds were sown in 8×8×7.5 cm pots filled with ˜300 g soil (10% peat, 20% top soil sieved through 2 mm mesh, 70% sand). All pots were watered to 70% of the soils water holding capacity. Once 50% of the control seeds had germinated, the seedlings were thinned to five per pot. Water/treatment was applied either once the seeds were sown (Day 1), after germination (Day 7), or on Day 1 and Day 7. Each treatment group was tested with and without base fertilizer (NPK 150-90-60), for a total of 9 different treatment groups, including water and boric acid application as controls. All treatments that received Protein Hydrolysate treatment saw 3.5× overall biomass growth compared to the water control and the control group being treated with the NPK fertilizer. The treatment group that received protein hydrolysate application on Day 7 only had the greatest overall growth.

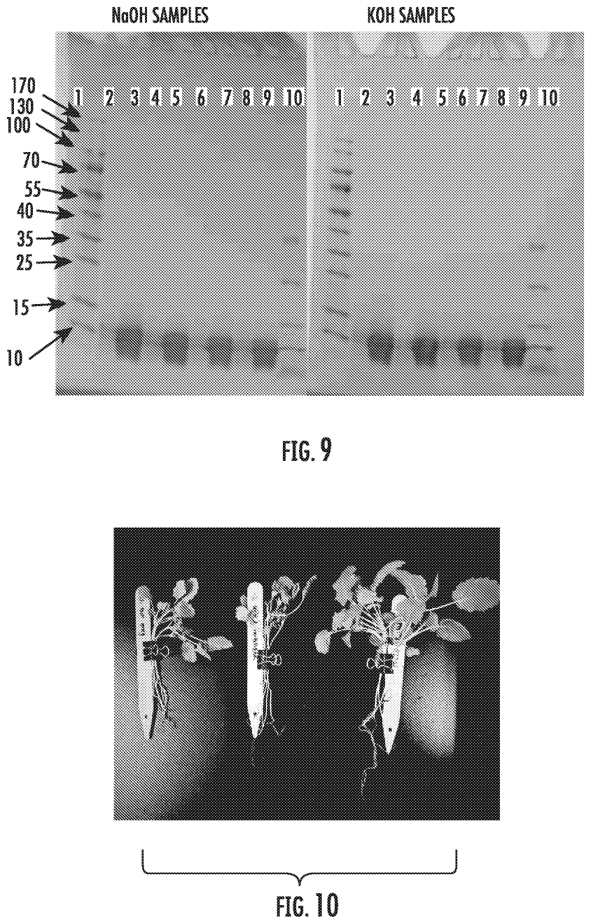

FIG. 10 shows, from left to right, turnips treated with: (a) water; (b) NPK fertilizer; (c) protein hydrolysate.

### Example 15: Demonstration of Biostimulant Effect of Protein Hydrolysate Produced Via Acidic and Alkaline Hydrolysis

Two protein hydrolysates were tested as biostimulants for lettuce plants. The first protein hydrolysate tested was produced using acidic hydrolysis as described herein. The second protein hydrolysate was produced using alkaline hydrolysis as described herein. Both were tested for their impact as a biostimulant on lettuce (Lactuca sativa). All procedures were as described in Xu, C., et al. (2017) HorTechnology 27(4):539-543. Lettuce seedlings were pre-germinated in peat moss before transplanting into pots containing sandy loam soil. The plants were watered and rotated 2×weekly, and grown under a 14 hr photo-period with 700 μmol photons/m² s light exposure. Water treatments and treatment with a commercial fish and seaweed hydrolysate were used as a controls to compare with the protein hydrolysates. Hoagland's solution, at 0.4 g Hoagland's salt mixture/L water, was used as a baseline nutrient solution for all treatments and applied to the soil the day the seedlings were transplanted. All treatments were normalized for nitrogen at the rate of 0.0165 g N/750 g starting soil and all results are reported as the average of 4 plants per treatment group.

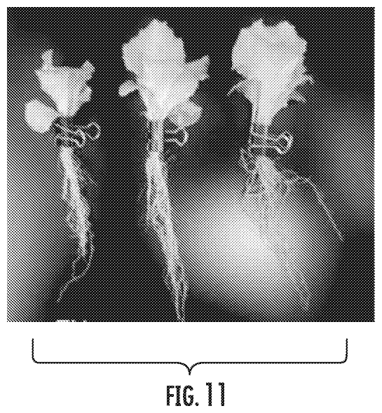

FIG. 11 shows, from left to right: Lactuca sativa treated with (a) a commercial fish and seaweed hydrolysate; (b) acid hydrolysate; (c) base hydrolysate.

Specific embodiments of the present disclosure have been described here in sufficient detail to enable those skilled in the art to practice the full scope of invention. However it is to be understood that many possible variations of the present disclosure, which have not been specifically described, still fall within the scope of the present invention and the appended claims. Hence these descriptions given herein are added only by way of example and are not intended to limit, in any way, the scope of the disclosed subject matter. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments disclosed herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, composition and/or method described herein. In addition, any combination of two or more such features, systems, articles, materials, kits, composition and/or methods, if such features, systems, articles, materials, kits, composition and/or methods are not mutually inconsistent, is included within the scope of the present invention.

All publications, patents, and patent applications cited herein are hereby incorporated by reference in their entireties for all purposes and to the same extent as if each individual publication, patent, or patent application were specifically and individually indicated to be so incorporated by reference.

1. A method for producing a protein hydrolysate comprising the steps of:
(a) culturing microorganism cells in a culture medium, thereby producing microbial biomass that comprises microbial protein;
(b) harvesting the microbial biomass;
(c) producing a biomass suspension composition from the harvested microbial biomass, wherein the biomass suspension composition comprises the microbial protein;
(d) adjusting the pH of the biomass suspension composition to a pH within a first target pH range of at least about 10, if needed, wherein the biomass suspension composition produced in step (c) or the pH adjusted suspension composition produced in step (d) is an alkaline suspension composition;
(e) heating the alkaline suspension composition to a first temperature of at least about 40° C. for a first time period of at least about 5 minutes, thereby producing an alkaline hydrolysate suspension that comprises hydrolyzed microbial protein.

2. The method of claim 1, further comprising:
(f) separating a liquid supernatant from solid material in the alkaline hydrolysate suspension, wherein the supernatant comprises soluble hydrolyzed microbial protein.

3. The method of claim 1, wherein step (e) comprises application of pressure of at least about 15 psi to the alkaline suspension for at least a portion of the first time period.

4. (canceled)

5. The method of claim 1, further comprising, after step (e):
(i) neutralizing the alkaline suspension composition by adding a neutralizing agent to the alkaline suspension composition, thereby forming a neutralized suspension composition, wherein the pH of the neutralized suspension composition is within a second target pH range of about 6.5 to about 9.5; and
(ii) adding a protease to the neutralized suspension composition at a second temperature of at least about 40° C. and a second time period of at least about 1 hour, thereby further hydrolyzing the microbial protein and forming a protease hydrolysate suspension that comprises hydrolyzed microbial protein.

6.-7. (canceled)

8. The method of claim 5, further comprising:
(f) separating a liquid supernatant from solid material in the protease hydrolysate suspension, wherein the supernatant comprises soluble hydrolyzed microbial protein.

9. A method for producing a protein hydrolysate comprising the steps of:
(a) culturing microorganism cells in a culture medium, thereby producing microbial biomass that comprises microbial protein;
(b) harvesting the microbial biomass;
(c) producing a biomass suspension composition from the harvested microbial biomass, wherein the biomass suspension composition comprises the microbial protein;
(d) adjusting the pH of the biomass suspension composition to a pH within a first target pH range of about 0.5 to about 3, if needed, wherein the biomass suspension composition produced in step (c) or the pH adjusted suspension composition produced in step (d) is an acidic suspension composition;
(e) heating the acidic suspension composition to a first temperature of at least about 40° C. for a first time period of at least about 5 minutes, thereby producing an acidic hydrolysate suspension that comprises hydrolyzed microbial protein.

10. The method of claim 9, further comprising:
(f) separating a liquid supernatant from solid material in the acidic hydrolysate suspension, wherein the supernatant comprises soluble hydrolyzed microbial protein.

11. The method of claim 9, wherein step (e) comprises application of pressure of at least about 15 psi to the acidic suspension for at least a portion of the first time period.

12. (canceled)

13. The method of claim 9, further comprising, after step (e):
(i) neutralizing the acidic suspension by adding a neutralizing agent to the acidic suspension composition, thereby forming a neutralized suspension composition, wherein the pH of the neutralized suspension composition is within a second target pH range of about 5 to about 8; and
(ii) adding a protease to the neutralized suspension composition at a second temperature of at least about 40° C. and a second time period of at least about 1 hour, thereby further hydrolyzing the microbial protein and forming a protease hydrolysate suspension that comprises hydrolyzed microbial protein.

14.-15. (canceled)

16. The method of claim 13, further comprising:
(f) separating a liquid supernatant from solid material in the protease hydrolysate suspension, wherein the supernatant comprises soluble hydrolyzed microbial protein.

17. The method of claim 1, wherein the microbial biomass produced in step (a) comprises at least about 0.1% w/v in the culture medium.

18. The method of claim 1, wherein the microbial biomass is subjected to lysis after step (b) and prior to step (c).

19. The method of claim 2, wherein the supernatant is dried to produce a dry or substantially dry composition that comprises the hydrolyzed microbial protein.

20.-23. (canceled)

24. The method of claim 1, wherein a chelating agent is added to the biomass suspension prior to step (e).

25. (canceled)

26. The method of any of claim 1, wherein a surfactant is added to the biomass suspension prior to step (e).

27. (canceled)

28. The method of claim 1, wherein a chaotropic agent is added to the biomass suspension prior to step (e).

29. (canceled)

30. The method of claim 1, wherein the microorganism cells are selected from Cupriavidus, Rhodococcus, Hydrogenovibrio, Rhodopseudomonas, Hydrogenobacter, Gordonia, Arthrobacter, Streptomycetes, Rhodobacter, and/or Xanthobacter cells.

31.-33. (canceled)

34. A protein hydrolysate composition comprising hydrolyzed microbial protein produced according to claim 1.

35.-42. (canceled)

43. A method of stimulating plant growth, comprising applying the composition of claim 34 to a seed, plant, or soil, wherein a plant grown in contact with the composition exhibits greater growth in the presence of the composition than in the absence of the composition.

44. The method according to claim 5, wherein step (d) and/or step (e) is replaced by a defatting step.
